(19) United States
(12) Patent Application Publication
(10) Pub. No.: US 2014/0178456 A1
(43) Pub. Date: Jun. 26, 2014

(54) METHODS AND COMPOSITIONS FOR TREATING TYPE 2 DIABETES AND RELATED CONDITIONS

(71) Applicant: UDAYA SANKAR DEVANABOYINA, FREMONT, CA (US)

(72) Inventor: UDAYA SANKAR DEVANABOYINA, FREMONT, CA (US)

(21) Appl. No.: 14/194,813

(22) Filed: Mar. 2, 2014

Related U.S. Application Data
(63) Continuation of application No. PCT/US2012/053191, filed on Aug. 30, 2012.

(51) Int. Cl. A61K 31/4439; A61K 9/00

(52) U.S. Cl. CPC A61K 31/4439; A61K 9/0024
USPC 424/426; 514/342; 424/423

(57) ABSTRACT

In some embodiments, there are provided methods and compositions for treating, preventing, or delaying the onset of type 2 diabetes and related disorders. The methods comprise administering a sustained release composition comprising a peroxisome proliferator-activated receptor (PPAR) agonist subcutaneously in a localized area of the subject. Slow release of the PPAR agonist in situ enhances the metabolic activity of subcutaneous adipose tissue, resulting in an increased ability of the tissue to clear excess glucose and lipid from the blood stream, while minimizing adverse side-effects of the agonist.

### FIELD

The present invention relates to certain novel peroxisome proliferator-activated receptor (PPAR) agonist compositions, and methods for their use in treating, preventing, or delaying the onset of type 2 diabetes and related disorders.

### BACKGROUND

The following description is provided to assist the understanding of the reader. None of the information provided or references cited is admitted to be prior art to the present invention.

Diabetes refers to a disease state or process derived from multiple causative factors and is characterized by elevated levels of plasma glucose (hyperglycemia) in the fasting state or after administration of glucose during a glucose tolerance test. Persistent or uncontrolled hyperglycemia is associated with a wide range of pathologies. Frank diabetes mellitus (e.g., fasting blood glucose levels above about 126 mg/dL) is associated with increased and premature cardiovascular disease and premature mortality, and is related directly and indirectly to various metabolic conditions, including alterations of lipid, lipoprotein, apolipoprotein metabolism and other metabolic and hemodynamic diseases. As such, the diabetic subject is at increased risk of macrovascular and microvascular complications. Such complications can lead to diseases and conditions such as coronary heart disease, stroke, peripheral vascular disease, hypertension, nephropathy, neuropathy, and retinopathy. Accordingly, therapeutic control and correction of glucose homeostasis is regarded as important in the clinical management and treatment of diabetes mellitus.

Diabetes is a major health problem, not only in the United States, but all over the world. Type 2 Diabetes is the most common form of diabetes and is characterized by disorders of insulin action and insulin secretion, either of which may be the predominant feature. In type 2 diabetes, or noninsulin dependent diabetes mellitus (NIDDM), subjects often produce plasma insulin levels comparable to those of nondiabetic subjects; however, the cells of subjects suffering from type 2 diabetes develop a resistance to the effect of insulin, even in normal or elevated plasma levels, on glucose and lipid metabolism, especially in the main insulin-sensitive tissues (muscle, liver and adipose tissue).

Insulin resistance is not associated with a diminished number of cellular insulin receptors but rather with a post-insulin receptor binding defect that is not well understood. This cellular resistance to insulin results in insufficient insulin activation of cellular glucose uptake, oxidation, and storage in muscle, and inadequate insulin repression of lipolysis in adipose tissue, and of glucose production and secretion in the liver. A net effect of decreased sensitivity to insulin is high levels of insulin circulating in the blood without appropriate reduction in plasma glucose (hyperglycemia). Hyperinsulinemia is a risk factor for developing hypertension and may also contribute to vascular disease.

Significant strides have been made in the management of type 2 diabetes in the last few decades. Changes to life-style, diet and exercise regimens have been successful to some extent in controlling the disease condition, however, compliance has been poor. As a result, the incidence of type 2 diabetes mellitus has continued to increase in the general population in U.S. and worldwide. Various treatments for type 2 diabetes are reviewed in the following references: Bastarrachea et al. (2008) Rev. Med. Chil. 136:107-17; Krentz et al. (2005) Drugs 65:385-41; Waugh et al. (2010) Health Technol. Assess. 14:1-248; Bennet et al. (2011) Agency for Healthcare Quality (AHRQ) Comparative Effectiveness Reviews Mar. Report No.: 11-EHC038-EF.

Some of the available treatment options, and their drawbacks are summarized below.

Increasing the plasma level of insulin by administration of sulfonylureas (e.g. tolbutamide and glipizide) or meglitinide, which stimulate the pancreatic beta-cells to secrete more insulin, and/or by injection of insulin when sulfonylureas or meglitinide become ineffective, can result in insulin concentrations high enough to stimulate insulin-resistance in tissues. However, dangerously low levels of plasma glucose can result from administration of insulin or insulin secretagogues (sulfonylureas or meglitinide), and an increased level of insulin resistance due to the even higher plasma insulin levels can occur. Additionally, patients develop resistance to insulin over the course of time, diminishing the effectiveness of the treatment.

Activators of Peroxisome Proliferator Activated Receptors (PPARs) have been used in the management of type 2 diabetes and related disorders. PPARs are members of the nuclear hormone receptor family of ligand regulated transcription factors (see Willson, et al. (2000) J. Med. Chem. 43:527-550). Three PPAR isoforms, PPARα, PPARγ and PPARδ, have been isolated from various mammalian species including humans. These receptors, as a class, form obligate heterodimers with their binding partner retinoic acid x receptor (RXR), and are activated by diet derived long chain fatty acids, fatty acid metabolites and/or by synthetic agents. PPARα regulates genes in the fatty acid synthesis, fatty acid oxidation, and lipid metabolism pathways (see Issenman and Green (1990) Nature 347:645-649; Torra et al. (1999) Current Opinion in Lipidology 10:151-159). The marketed PPARα agonists, such as fenofibrate and gemfibrozil, lower plasma lipids in mammals including humans (see Balfour et al. (1990) Drugs. 40:260-290; Frick et al. (1987) New Eng. J. Med. 317:1237-1245; Rubins et al. (1999) New Eng. J. Med. 341:410-418). PPARγ has been demonstrated to regulate pre-adipocyte recruitment and differentiation into mature adipocytes. The role of the relatively more ubiquitously expressed PPARδ (also known as PPAR13, herein referred to as PPARδ(β)) isoform has been unclear although it is known that: (1) PPARδ(β) is present in pre- and mature adipocytes, and (2) it is activated by fatty acids and fatty acid metabolites (see, Zhang et al. (1996) Mol. Endocrinology. 10:1457-1466; Berger et al. (1999) J. Biol. Chem. 274:6718-6725; Bastie et al. (1999) J. Biol. Chem. 274:21920-21925).

Activators of PPARγ promote lipid storage in adipocytes and act as insulin sensitizing anti-diabetic agents (see Lehmann et al. (1995) J. Biol. Chem. 270:12953-12956; Nolan et al. (1994) New. Eng. J. Med. 331:1188-1193; Inzucchi et al. (1998) New Eng. J. Med. 338:867-872). The glitazones (i.e., 5-benzylthiazolidine-2,4-diones) are a class of compounds that have proven useful for the treatment of type 2 diabetes. The currently marketed glitazones are agonists of the peroxisome proliferator activated receptor (PPAR), primarily the PPARγ subtype. These agents increase insulin sensitivity in muscle, liver and adipose tissue in several animal models of type 2 diabetes, resulting in partial or complete correction of the elevated plasma levels of glucose without occurrence of hypoglycemia. PPARγ agonism is generally believed to be responsible for the improved insulin sensitization that is observed with the glitazones.

The U.S. Food and Drug Administration has approved two glitazone drugs, Avandia® (Rosiglitazone) and Actos® (Pioglitazone), for the treatment of insulin resistance. However, serious potential adverse side effects have been reported, including myocardial infarction, congestive heart failure, stroke, macular edema, bone fracture, osteoporosis, fluid retention, and bladder cancer. Another PPARγ agonist, troglitazone, (Rezulin®) was withdrawn from the market following reports of drug-induced hepatitis.

Hence, despite the development of several biological, pharmaceutical and medical device-based treatment options which have been successfully developed over the past several years to treat type 2 diabetes and related disorders, as these are chronic life-long disease conditions, and as most of the drugs carry some adverse effects, a significant need exists for effective new treatment options.

### SUMMARY

The following presents a simplified summary of the invention in order to provide a basic understanding of some aspects of the invention. This summary is not an extensive overview of the invention. It is intended to neither identify key or critical elements of the invention nor delineate the scope of the invention. Rather, the sole purpose of this summary is to present some concepts of the invention in a simplified form as a prelude to the more detailed description that is presented hereinafter.

In some embodiments, there are provided methods for treating a mammalian subject with a condition selected from the group consisting of type 2 diabetes, and related disorders such as hyperlipidemia, cardiovascular diseases, hyperglycemia, and insulin resistance, obesity, gastro-intestinal, reproductive and various metabolic disorders, wherein the methods comprise exposing subcutaneous adipose tissue of the subject in situ to a PPAR agonist.

In some embodiments, there are provided herein methods and formulations for preventing or treating type 2 diabetes and related disorders in a subject. The methods comprise exposing subcutaneous adipose tissue in situ in the subject to a PPAR agonist. The concentration and duration of exposure are sufficient to effect a change in the morphology and molecular markers of the subcutaneous adipose tissue which leads to a lowering of blood glucose and lipid levels in the subject, thereby preventing or treating type 2 diabetes and related disorders in the subject.

In some embodiments, there are provided methods for treating a subject with a condition selected from the group consisting of type 2 diabetes and related diseases. The methods comprise administering a sustained release composition comprising a PPAR agonist subcutaneously in a localized area of the subject, wherein the area is selected from at least one of abdomen, chest, breast, flank, inguinal region, back, trunk, hip, suprascapular region, leg, arm, thigh, buttock, and combinations thereof. Subcutaneous adipose tissue in the area is exposed to the agonist in situ. In some embodiments, the agonist is selected from the group consisting of the thiazolidinedione or non-thiazolidinedione class of PPAR agonists. Non-limiting examples of the agonist include rosiglitazone, ciglitazone, troglitazone, englitazone, pioglitazone, muraglitazar, ragaglitazar, naveglitazar, and mixtures thereof. In some embodiments, said PPAR agonist is a nonthiazolidinedione

In some embodiments, the PPAR agonist in a sustained release composition as disclosed herein is a PPARγ agonist, as exemplified by pioglitazone and rosiglitazone. The sustained release composition is formulated to release sufficient PPAR agonist to confer a therapeutic effect, but at a low blood plasma concentration of the PPAR agonist, thereby minimizing systemic exposure to the PPAR agonist and substantially avoiding potential adverse side-effects. In some embodiments, a composition for subcutaneous administration, as described herein, is formulated to release a PPAR agonist in a daily dose that is a fraction of an effective oral daily dose of said agonist, and wherein said daily dose from subcutaneous administration of said composition provides a therapeutic effect substantially equivalent to that of said oral daily dose.

In some embodiments, the disclosed methods are effective to decrease serum glucose levels of a mammal to normal levels. In some embodiments, the methods are effective to decrease serum triglyceride and lipid levels of a mammal to normal levels.

In some embodiments there are provided methods and compositions for treating a condition, or a combination of conditions, selected from hyperglycemia, low glucose tolerance, insulin resistance, obesity, abdominal obesity, lipid disorders, dyslipidemia, hyperlipidemia, hypertriglyceridemia, hypercholesterolemia, low HDL levels and/or high LDL levels, atherosclerosis and its sequelae, fluid retention, vascular restenosis, pancreatitis, neurodegenerative disease, retinopathy, nephropathy, neuropathy, Syndrome X, gastrointestinal motility disorders, fertility and reproductive disorders, and other conditions where insulin resistance or hyperglycemia is a component, in a subject in need thereof, comprising administering to said subject a PPAR agonist in accordance with the methods and compositions disclosed herein.

In some embodiments, there are provided methods for treating a mammalian subject with a condition selected from the group consisting of type 2 diabetes, hyperlipidemia, and cardiovascular disease, wherein said methods comprise administering a sustained release composition into a subcutaneous space in a localized area of said subject; wherein said composition comprises a peroxisome proliferator-activated receptor (PPAR) agonist; wherein said composition releases said agonist directly into said subcutaneous space; wherein subcutaneous adipose tissue in said space is exposed in situ to a therapeutically effective amount of said agonist; and, whereby systemic exposure in said subject to said agonist is minimized.

In some embodiments, the agonist is selected from the group consisting of rosiglitazone, ciglitazone, troglitazone, englitazone, pioglitazone, muraglitazar, ragaglitazar, naveglitazar, and mixtures thereof.

In some embodiments, the composition is formulated to release said agonist in a total daily amount that is in the range of about one tenth to about one thousandth of a therapeutically effective oral daily dose of said agonist, and wherein said total daily amount of said agonist provides a therapeutic effect substantially equivalent to that of said oral daily dose.

In some embodiments, the therapeutically effective amount elicits an effect selected from at least one of improved glycemic control, euglycemia, and an improved lipid profile.

In some embodiments, a ratio of said PPAR agonist concentration in said subcutaneous space to a steady-state plasma concentration of said PPAR agonist plus its active metabolites in said subject is in the range of about 2 to about 10000.

In some embodiments, said localized area is selected from at least one of abdomen, chest, arm, leg, breast, inguinal region, back, hip, flank, suprascapular region, thigh, buttock, and combinations thereof.

In some embodiments, said tissue is exposed to said agonist for a duration sufficient to detectably effect an alteration in a morphological feature of said tissue, wherein said feature is selected from at least one of an increase the number of adipocytes within said tissue, an increase in the number of mitochondria per adipocyte within said tissue, and the appearance of a multilocular morphology of adipocytes within said tissue; wherein the appearance of said beneficial effect coincides with said alteration of said morphological feature.

In some embodiments, said tissue is exposed to said agonist for a duration sufficient to detectably increase the amount of UCP-1 in said tissue and/or to detectably increase the oxygen uptake of said tissue.

In some embodiments, said agonist is administered at a dose at which an adverse side effect due to said PPAR agonist is substantially undetectable in said subject, wherein said side effect is selected from at least one of cardiovascular disease, osteoporosis, increased susceptibility for bone fracture, adipogenesis in bone marrow, bladder cancer, and hepatitis.

In some embodiments, said agonist is administered at a dose at which an adverse side effect due to said PPAR agonist is substantially undetectable in said subject, wherein said side effect is selected from at least one of myocardial infarction, stroke, macular edema, fluid retention, cardiac hypertrophy, atherosclerosis, and congestive heart failure.

In some embodiments, said agonist is rosiglitazone or pioglitazone and wherein said side effect comprises cardiovascular disease, osteoporosis, increased susceptibility for bone fracture, fluid retention, and adipogenesis in bone marrow.

In some embodiments, said agonist is pioglitazone and wherein said side effect is bladder cancer.

In some embodiments, said agonist is troglitazone and said side effect is hepatitis.

In some embodiments, said subject is able to achieve an average preprandial plasma glucose concentration in the range of about 72 mg per deciliter to about 108 mg per deciliter due to said treatment.

In some embodiments, said subject is able to achieve an average bedtime plasma glucose values between about 110 mg per deciliter to about 150 mg per deciliter.

In some embodiments, said subject is able to achieve a 2-hour postprandial blood glucose in the range of about 90 mg per deciliter to about 144 mg per deciliter.

In some embodiments, said subject is able to achieve an HbA_{1c} value less than about 7%.

In some embodiments, said sustained release composition comprises said agonist coated onto a biodegradable or non-biodegradable scaffold and wherein the scaffold is inserted into the subcutaneous space via a surgical procedure.

In some embodiments, said subcutaneous adipose tissue comprises white adipose tissue.

In some embodiments, said agonist is selected from at least one of a PPARα agonist, a PPARγ agonist, a PPARδ agonist, a PPARβ agonist, a PPARδ(β))) agonist, a dual PPAR agonist, a pan PPAR agonist, and combinations thereof.

In some embodiments, said agonist is a thiazolidinedione. In some embodiments, said agonist is a nonthiazolidinedione.

In some embodiments, said agonist is rosiglitazone, and wherein the amount of rosiglitazone released results in an AUC₀₋₂₄ₕ of rosiglitazone which does not exceed about 300 ng-h/mL in plasma of said subject.

In some embodiments, said agonist is pioglitazone, and wherein the amount of pioglitazone released results in an AUC₀₋₂₄ₕ of pioglitazone and its active metabolites which do not exceed about 10 μg-h/mL in plasma of said subject.

In some embodiments, said agonist is rosiglitazone and wherein said rosiglitazone is released at a rate of about 0.0001 μg per day to about 1000 μg per day.

In some embodiments, said agonist is pioglitazone and wherein said pioglitazone is released at a rate of about 0.0001 μg per day to about 10 mg per day.

In some embodiments, said administering results in a weight gain of said subject of about 0.05 kg to about 10 kg due to an increase in mass of said subcutaneous adipose tissue.

In some embodiments, said peroxisome proliferator-activated receptor agonist is administered to the body of the subject such that said mass is substantially symmetrically distributed.

In some embodiments, the subcutaneous adipose tissue comprises white adipose tissue, and wherein said white adipose tissue maintains contact with the agonist at a concentration and over a duration sufficient to maintain a metabolically active morphology in said white adipose tissue.

In some embodiments, there are provided methods for preventing or treating type 2 diabetes and related disorders in a subject, the methods comprising: exposing subcutaneous adipose tissue of said subject in situ to peroxisome proliferator-activated receptor agonist at a sufficient level and over a sufficient duration to activate brown adipocyte-like differentiation in said adipose tissue but wherein substantially no adverse effect due to said agonist is detectable in said subject, wherein the differentiated subcutaneous adipose tissue increased energy expenditure.

In some embodiments, there are provided methods comprising: exposing subcutaneous adipose tissue in situ in a subject to a peroxisome proliferator-activated receptor agonist, wherein said exposing is sufficient to increase and maintain an increase in the quantity of a subset of adipocytes which comprises brite adipocytes in said tissue.

In some embodiments, there are provided methods for preventing or treating atherosclerosis in a non-diabetic subject, the methods comprising: exposing subcutaneous adipose tissue of said subject in situ to a peroxisome proliferator-activated receptor agonist at a sufficient concentration to activate differentiation in said subcutaneous adipose tissue, wherein the differentiated subcutaneous adipose tissue has enhanced energy expenditure.

In some embodiments, there are provided methods for preventing or treating hyperlipidemia in a non-diabetic subject, the methods comprising: exposing subcutaneous adipose tissue of said subject in situ to a peroxisome proliferator-activated receptor agonist at a sufficient concentration to activate differentiation in said subcutaneous adipose tissue, wherein the differentiated subcutaneous adipose tissue has enhanced energy expenditure,

In some embodiments, there are provided compositions comprising rosiglitazone formulated to release said rosiglitazone at a rate in the range of about 1 pg per day to about 1 mg per day when said composition is administered into a subcutaneous space comprising white adipose tissue in a mammalian subject. In an active ingredient as disclosed herein, such as rosiglitazone, is coated onto a biodegradable or non-biodegradable scaffold.

In some embodiments, there are provided compositions comprising pioglitazone formulated to release said pioglitazone at a rate in the range of about 1 pg per day to about 1 mg per day when said composition is administered into a subcutaneous space comprising white adipose tissue in a mammalian subject. In some embodiments, said pioglitazone is coated onto a biodegradable or non-biodegradable scaffold.

In some embodiments, there are provided unit dosage forms and kits comprising compositions as disclosed.

### DESCRIPTION

The disclosed methods and pharmaceutical compositions are useful in treating type 2 diabetes and related disorders in a mammal.

It is disclosed herein that even lower levels of a PPAR agonist than previously suspected lower or substantially eliminate the symptoms of type 2 diabetes and related disorders. By use of the disclosed methods and formulations, which involve targeted administration to subcutaneous adipose tissue in situ over an extended time period, remarkably low doses of PPAR agonist produce therapeutic results. Advantageously, the use of these low doses results in low plasma levels of the agonist, which thereby substantially minimizes the reported adverse side effects, such myocardial infarction, stroke, macular edema, bone fracture, osteoporosis, bladder cancer, and hepatitis.

Without wishing to be bound by theory, in some embodiments, the administration of a PPAR agonist by the methods as disclosed herein exposes subcutaneous adipose tissue in situ to a low and substantially continuous concentration of the PPAR agonist which leads to an increase of the adipocyte population of the adipose tissue and also leads to a change in the adipocyte morphology and metabolic profile. Such changes include an increase in the number of mitochondria per cell, an increase in the number of lipid vacuoles per cell, and a decrease in the size of intracellular lipid vacuoles, compared to the untreated controls. The morphological changes are accompanied by molecular changes in the expression of various molecular markers that play an important role in the beta-oxidation pathway. White adipose tissue exposed to a PPAR agonist as disclosed herein becomes more metabolically active and express a brown adipose tissue-like (i.e., brite adipose tissue) phenotype (see, e.g., Petrovic et al. (2010) J. Biol. Chem. 285:7153-7164). PPAR agonists as described herein may also alter and maintain the morphology of the adipocytes through mechanisms other than PPAR agonism (see, e.g., Duan et al. (2010) Diabetologia 53:1493-1505). Adipocytes exposed to a PPAR agonist as disclosed herein become more metabolically active and acquire the ability to act as a metabolic “sink” for clearing excess glucose and lipids from the blood stream.

It is to be appreciated that certain aspects, modes, embodiments, variations and features of the invention are described below in various levels of detail in order to provide a substantial understanding of the present invention.

Before describing the present disclosure in detail, it is to be understood that this disclosure is not limited to specific compositions or method steps, as such can vary. It is also to be understood that the terminology used herein is for the purpose of describing particular embodiments only, and is not intended to be limiting. Methods recited herein can be carried out in any order of the recited events that is logically possible, as well as the recited order of events. Where a range of values is provided, it is understood that each intervening value, between the upper and lower limit of that range, and any other stated or intervening value in that stated range, is encompassed within the description. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included in the present disclosure. Also, it is contemplated that any optional feature of the disclosed variations described can be set forth and claimed independently, or in combination with any one or more of the features described herein.

All literature and similar materials cited in this application, including but not limited to patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

The publications discussed herein are provided solely for their disclosure prior to the filing date of the present application. Nothing herein is to be construed as an admission that the present disclosure is not entitled to antedate such publication by virtue of prior invention. Further, the dates of publication provided may be different from the actual publication dates, which may need to be independently confirmed.

The practice of the present disclosure will employ, unless otherwise indicated, conventional techniques which are within the skill of the art. Such techniques are explained fully in the literature.

Unless specifically defined herein, all terms used herein have the same meaning as they would to one skilled in the art of the present disclosure. Various methods, devices and materials similar or equivalent to those described herein can be used in the practice or testing of the disclosed methods.

It must be noted that, as used in the specification and the appended claims, the singular forms “a,” “an” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Unless otherwise indicated, a percentage refers to a percentage by weight (i.e., % (W/W)).

As used herein, “about” will be understood by persons of ordinary skill in the art and will vary to some extent depending upon the context in which it is used. “About” will mean up to plus or minus 10% of the enumerated value.

As used herein, the “administration” of an agent or drug to a subject includes any route of introducing or delivering to a subject a compound to perform its intended function. Administration includes self-administration and the administration by another.

As used herein, “subcutaneous delivery” means directly depositing underneath the skin, by use of an applicator such as a needle, a cannula, a multi-needle array, an energy-based delivery system capable of subcutaneous delivery, a pressure-based delivery system capable of subcutaneous delivery, a needleless delivery system capable of subcutaneous delivery, or a similar medical device. A sustained release formulation as disclosed herein, may be subcutaneously delivered within, and/or in the vicinity of, adipose tissue located in the subcutaneous space.

As used herein, the terms “controlled”, “extended”, “sustained”, “continuous” or “prolonged” release of active agents, such as PPAR agonists disclosed herein, will collectively be referred to as “sustained release” and include continuous or discontinuous, intermittent, linear or non-linear release.

As used herein, “subcutaneous adipose tissue” refers to adipose tissue located within the subcutaneous space at fat depots such as abdomen, chest, breast, flank, inguinal region, back, trunk, hip, suprascapular region, leg, arm, thigh, buttock, which is metabolically distinct from visceral fat. Subcutaneous adipose tissue can be white fat, brown fat, brown-like fat, brite fat, or other subtypes.

As used herein, “pharmaceutically-acceptable” means that the compound(s), carrier(s), or product(s), which the term describes are suitable for subcutaneous delivery without undue toxicity, incompatibility, instability, irritation, allergic response, and the like.

The term “subject” as used herein refers to a member of any vertebrate species. The methods of the presently disclosed subject matter are particularly useful for warm-blooded vertebrates. Provided herein is the treatment of mammals such as humans, as well as those mammals of importance due to being endangered, of economic importance (animals raised on farms for consumption by humans) and/or social importance (animals kept as pets or in zoos) to humans. In some embodiments, the subject is a human.

As used herein, the terms “treating” or “treatment” or “alleviation” refers to both therapeutic treatment and prophylactic or preventative measures, wherein the object is to prevent or slow down (lessen) the targeted pathologic condition or disorder.

As used herein, “prevention” or “preventing” of a disorder or condition refers to a compound or method that, in a statistical sample, reduces the occurrence of the disorder or condition in the treated sample relative to an untreated control sample, or delays the onset or reduces the severity of one or more symptoms of the disorder or condition relative to the untreated control sample.

The term “related diseases” in reference to type 2 diabetes includes hyperglycemia, low glucose tolerance, insulin resistance, hyperinsulinemia, obesity, abdominal obesity, lipid disorders, dyslipidemia, hyperlipidemia, hypertriglyceridemia, hypercholesterolemia, low HDL levels and/or high LDL levels, atherosclerosis, atherosclerosis and its sequelae, fluid retention, vascular restenosis, pancreatitis, neurodegenerative disease, retinopathy, nephropathy, neuropathy, Syndrome X, gastrointestinal motility disorders, fertility and reproductive disorders, and other conditions where insulin resistance or hyperglycemia may be a component.

The conditions, diseases, and maladies collectively referenced to as “Syndrome X” or Dysmetabolic Syndrome are detailed in Johannsson (1997) J. Clin. Endocrinol. Metab. 82:727-734 and other publications.

The conditions, diseases and maladies collectively referred to as “diabetic complications” include coronary heart disease, stroke, peripheral vascular disease, hypertension, nephropathy, neuropathy, and retinopathy, and other known complications of diabetes.

“Adverse effects” as used herein, means those physiological effects to various systems in the body such as cardiovascular systems, nervous system, digestive system, and the body as a whole, which cause pain and discomfort to the individual subject.

Body mass index (BMI) is a measure of body fat based on height and weight that applies to adult men and women. BMI categories include the following: Underweight=<18.5; Normal weight=18.5-24.9; Overweight=25-29.9; Obesity=BMI of 30 or greater.

“Implant” means a sustained release drug delivery system. The implant may be comprised of a biocompatible polymer or ceramic material which contains or which can act as a carrier for a molecule with a biological activity. The implant can be, injected, inserted or implanted into a human body.

As used herein, “treatment” means any manner in which one or more of the symptoms of a disease or disorder are ameliorated or otherwise beneficially altered. Treatment also encompasses any pharmaceutical use of the compositions herein.

Formulations and Uses

Any suitable formulation may be used in the present methods as long as it can release the PPAR agonist in a sustained release manner as disclosed herein.

In some embodiments, there are disclosed herein uses of a pharmaceutical composition comprising one or more PPAR agonists and/or a pharmaceutically acceptable salt thereof, together with one or more pharmaceutically acceptable carriers thereof and, optionally, other therapeutic and/or prophylactic ingredients. In some embodiments, a PPAR agonist as used herein may comprise a single isomer, a mixture of isomers, or a racemic mixture of isomers; a solvate, clathrate, or polymorph; or a prodrug or metabolite thereof.

As used herein the term, “pharmaceutically acceptable derivatives” refers to salts, esters, enol ethers, enol esters, acetals, ketals, orthoesters, hemiacetals, hemiketals, acids, bases, solvates, hydrates or prodrugs thereof of a compound. Such derivatives may be readily prepared by those of skill in this art using known methods for such derivatization. The compounds produced may be administered to animals or humans without substantial toxic effects and either are pharmaceutically active or are prodrugs. As used herein, the term “prodrug” refers to a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes.

PPAR agonists can be formulated as pharmaceutically acceptable salts (e.g., acid addition salts) and/or complexes thereof. The preparation of such salts can facilitate the pharmacological use by altering the physical-chemical characteristics of the composition without preventing the composition from exerting its physiological effect. Examples of useful alterations in physical properties include melting point and solubility. In practice the use of the salt form is substantially equivalent to use of the base form. The compounds of the present disclosure are useful in both free base and salt form, with both forms being considered within the scope of the present disclosure.

Pharmaceutically acceptable salts include acid addition salts such as those containing sulfate, hydrochloride, phosphate, sulfamate, acetate, citrate, lactate, tartrate, maleate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethane sulfonic acid, benzene sulfonic acid, p-toluenesulfonic acid, cyclohexyl sulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

Examples of a “PPAR agonist” include a PPARα agonist, a PPARγ agonist, a dual PPAR agonist, a PPARα/γ dual agonist, a pan PPAR agonist, and a PPARδ(β)) agonist.

Non-limiting examples of PPAR agonists useful in the present methods and compositions include PPARα agonists, PPARγ agonists, PPARδ agonists, PPARβ agonists, PPARδ(β)) agonists, dual agonists, and pan agonists. For purposes of simplifying the description of the disclosure, and not by way of limitation, compositions comprising PPARγ agonists will primarily be described herein, it being understood that essentially all PPAR agonists are intended to be included within the scope of this invention.

As used herein, “agonist of the peroxisome proliferator-activated receptor-gamma” or PPARγ agonist means a molecule, or a mixture of agents containing such a molecule (e.g., a botanical extract), that directly interacts with the PPARγ protein, and stimulates its interaction with retinoid X receptors and/or its target genes, to produce a physiological effect.

Non-limiting examples of PPARγ agonists include thiazolidinedione oral anti-diabetic agents and other insulin sensitizers (which have an insulin sensitivity effect in type 2 diabetes subjects) such as troglitazone (Warner-Lambert's Rezulin®, disclosed in U.S. Pat. No. 4,572,912), rosiglitazone (SKB), pioglitazone (Takeda), Mitsubishi's MCC-555 (disclosed in U.S. Pat. No. 5,594,016), Glaxo-Welcome's GL-262570, englitazone (CP-68722, Pfizer), darglitazone (CP-86325, Pfizer), isaglitazone (MIT/J&J), JTT-501 (JPNT/P&U), L-895645 (Merck), R-119702 (Sankyo/WL), NN-2344 (Dr. Reddy/NN), ciglitazone, YM-440 (Yamanouchi), and mixtures thereof.

In some embodiments, a PPAR agonist useful in the present methods is a nonthiazolidinedione, non-limiting examples of which include: GW1929 (Lu et al (2010) Eur. J. Pharmacol. 636:192-202), FK614 (Minoura et al. (2005) Eur. J. Pharmacol. 519:1182-190), WY14653 (Berger et al. (1999) J. Biol. Chem. 274:6718-6725), and T33 (Hu et al. (2006) Acta Pharmacologica Sinica 27:1346-1352), and the like.

In some embodiments, a PPARγ agonist includes botanical and natural extracts which are known to enhance adipocyte differentiation. Such extracts, or fractions thereof, might be known activators of the PPARγ pathway (e.g. Pulpactyl, an extract from Artemisia abrotanum; Southernwood), or might be known for their ability to enhance lipid production in experimental systems or in humans (e.g. Einkorn, an extract from Triticum monococcum).

In some embodiments, compositions useful in methods disclosed herein contain formulations suitable for subcutaneous application. In some embodiments, the composition contains an agonist of PPARγ and a pharmaceutically acceptable carrier.

In some embodiments, subcutaneous administration of a PPAR agonist is carried out by the use of depot injection, an implant, as a nanomaterial, nanostructure, nanofiber, nanowire, nanoparticle, microsphere, quantum dot, nanotube, dendrimer, nanocystal, or nanobot, rechargeable or biodegradable device, sustained release polymeric device, infusion, pump, infusion pump, continuous infusion, sustained release formulation, bound to a polymer matrix, and sustained release patch.

In some embodiments, when a PPAR agonist is administered as a pharmaceutical to a subject, it can be given as a pharmaceutical composition containing, in some embodiments, 0.1 to 99.5% or in some embodiments, 0.5 to 90%, of PPAR agonist in combination with a pharmaceutically acceptable carrier. In some embodiments, a PPAR agonist constitutes from about 0.0000001% to about 50%, by weight of the composition, from about 0.00001% to about 20%, by weight of the composition, from about 0.001% to about 10% by weight of the composition, and, in some embodiments, from about 0.01% to about 1% by weight of the composition.

In some embodiments, formulations may include scaffold materials (Flynn et al. (2008) Organogenesis 4:278-235) as exemplified by: synthetic scaffolds such as poly(lactic-co-glycolic)-acid-scaffolds, polyglycolic acid scaffolds, polytetrafluoroethylene scaffolds, poly(ethylene glycol)-diacrylate hydrogels, polyethylene terephthalate scaffolds, Matrigel™, collagen scaffods, HYAFF® scaffolds, alginate scaffolds, fibrin scaffolds, and decellularized matrices. In some embodiments, a PPAR agonist may be coated onto a biodegradable or non-biodegradale scaffold and optimized to release the drug over an extended period. The scaffold may be inserted into the subcutaneous space via a surgical procedure.

Formulations as disclosed herein may also include enzymes that dissociate connective tissue or other tissues, such as, e.g., hyaluronidase and trypsin.

One or more pharmaceutically acceptable carriers may be present in a formulation of the present disclosure. Pharmaceutically-acceptable agents for subcutaneous delivery are well-known; examples of descriptions of such agents include: Handbook on Injectable Drugs, 14th Edition published by the American Society of Health-System Pharmacists (ASHP); and the “inactive ingredients for approved drug products” database published online by the Center for Drug Evaluation and Research (CDER) at the U.S. Food and Drug Administration (FDA). Other pharmaceutically acceptable carriers and their formulation are described in standard formulation treatises, e.g., Remington's Pharmaceutical Sciences, 17^{th} Edition (Mack Publ., Co. 1985) or later editions.

In some embodiments, carriers include, but are not limited to, water, ethanol, isopropanol, 1,2-propanediol, glycerin, benzyl alcohol, dimethylisosorbide, triacetin, glycol ethers, propylene glycol and polyethylene glycol (PEG). Some embodiments of solvents include PEG having an average molecular weight between about 200 and about 400, castor oil, triacetin, dimethylisosorbide, ethanol, and water, and combinations thereof.

Various compounds may be added to the formulation to alter osmolarity and/or pH to acceptable levels. These include, but are not limited to, mannitol, sucrose, calcium chloride, sodium chloride, sodium phosphate monobasic, sodium phosphate dibasic, sodium hydroxide, and hydrochloric acid.

A surfactant may be added to the composition. Exemplary surfactants include nonionic surfactants such as polysorbates (e.g. polysorbates 20, 80, such as Tween® 20, Tween® 80) or poloxamers (e.g., poloxamer 188). The amount of surfactant added is such that it reduces aggregation of the formulation and/or minimizes the formation of particulates in the formulation, without reducing the biological activity. The surfactant may be present in the formulation in an amount from about 0.001% to about 0.5%, from about 0.005% to about 0.1%, or from about 0.01% to about 0.05%.

In some embodiments, subcutaneous compositions are provided which may be formulated as emulsions. If the carrier is an emulsion, from about 1% to about 10% (or from about 2% to about 5%) of the carrier can be made up of one or more emulsifiers. Emulsifiers may be nonionic, anionic or cationic. Suitable emulsifiers may be found in, for example, the 2008 International Cosmetic Ingredient Dictionary and Handbook, 12th Edition published by the Personal Care Products Council.

In some embodiments, the disclosed compositions may be formulated as a gel (e.g., an aqueous gel using a suitable gelling agent(s)). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprises between about 0.1% and 5%, by weight, of such gelling agents. Non-limiting examples of suitable copolymers include glycolide, betapropiolactone, tetramethylglycolide, betabutyrolactone, tetramethylglycolide, f3-butyrolactone, gammabutyrolactone, pivalolactone, intramolecular cyclic esters of alphahydroxybuteric acid, alphahydroxy, isovaleric acid, alphahydroxycaproic acid, alphahydroxy ethylbuteric acid, alphahydroxy isocaproic, alphahydroxy betamethyl valeric acid, alphahydroxy heptonic acid, alphahydroxy octanic acid, alphahydroxy deccanoic acid, alphahydroxy myristic acid, alphahydroxy stearic acid, alphahydroxy ligocenic acid, polyglycolic acids, and betaphenol lactic acid.

In some embodiments, a PPAR agonist can be coupled with soluble polymers as drug carriers. Such polymers can include polyvinylpyrrolidone, pyran copolymer, polyhydroxylpropylmethacrylamidephenol, polyhydroxyethylaspartamidephenol, or polyethyleneoxide-polylysine substituted with palmitoyl residues. In some embodiments, a PPAR agonist can be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels. Other pharmaceutical compositions for administration are discussed, for example, in Remington's Pharmaceutical Sciences (1985).

In some embodiments, formulations and dosage compositions can be prepared by mixing the ingredients following generally accepted procedures. For example, the selected components may be simply mixed in a blender or other standard device to produce a concentrated mixture which may then be adjusted to the final concentration and viscosity by the addition of water or thickening agent and possibly a buffer to control pH or an additional solute to control tonicity.

In some embodiments, a formulation can be a liquid, solid, or semi-solid depot, slow, or sustained release formulation capable of releasing a PPAR agonist subcutaneously over essentially any desired time period as disclosed herein.

In some embodiments, a PPAR agonist formulation is prepared with carriers that will protect the compound against rapid elimination from the body, such as a sustained release formulation, including implants and microencapsulated delivery systems. In some embodiments, biodegradable, biocompatible polymers can be used, such as ethylene vinyl acetate, polyanhydrides, polyglycolic acid, collagen, polyorthoesters, and polylacetic acid. In some embodiments, non-biodegradable materials may be used. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art (see, e.g., U.S. Pat. Nos. 4,522,811, 6,306,423 and 6,312,708; U.S. Published Pat. Application No. 20110123618; Lichtenberg et al. (1988) Methods Biochem. Anal. 33:337-462; Anselem et al. (1993) Liposome Technology, CRC Press).

In some embodiments, a delivery system similar to, or the same as, an Alza osmotic system may be employed. These systems employ two layers, a drug layer and an osmotically driven displacement layer all surrounded by a water permeable/drug impermeable membrane with an exit passage in this membrane for the drug.

In some embodiments, formulations as disclosed herein are bioabsorbable, that is, can be totally absorbed by the host's body. Because of this feature, the implants need not be removed from the subject once it is implanted, since it is eventually totally absorbed by the subject's body, and thus eliminates the need for surgical removal of the implant. In some embodiments, formulations as disclosed herein are not bioabsorbable

A formulation as described herein may further contain other materials such as collagen, cross-linked collagen, hyaluronic acid, poly lactic acid, calcium hydroxyl apatite, polymers, cells, minced tissues, autologous transplanted cells or tissues, being intact or fragmented, gelatin, or the mixtures thereof.

As used herein, “cross-linked collagen” means a polymer composite of collagen molecules that are connected together. Cross-links are covalent bonds linking one polymer chain to another, which are formed by chemical reactions that are initiated by heat and/or pressure, or by the mixing of an unpolymerized or partially polymerized unit with specific chemicals called crosslinking reagents. Crosslinking inhibits the close packing of polymer chains and prevents the formation of crystalline regions. A cross-linked biological structure such as cross-linked collagen has restricted molecular mobility which limits the extension of the polymer material, and is less prone to degradation than the collagen monomer. Suitable cross-linked collagens for use in the present methods include, but are not limited to, collagen molecules of natural or synthetic sources that are cross-linked by e.g., heat, solvents, organic agents, coagulation agents, sugars, glycosaminoglycans, glutaraldehydes, and the like.

As used herein, “sugar cross-linked collagen” means collagen molecules that are chemically connected by reacting with sugars. One non-limiting example of sugar cross-linked collagen is a collagen cross-linked by the Glymatrix™ technology, which is based on a non-enzymatic glycation process. This cross-linking technology utilizes D-ribose as a cross linking agent. In some embodiments, the sugar cross-linked collagen constitutes from about 1% to about 10%, of a formulation, from about 1.5% to about 8%, by weight, of a formulation, from about 2.5% to about 4.5% by weight of a formulation.

Formulations as disclosed herein may, where appropriate, be conveniently presented in a discrete unit dosage form and may be prepared by any of the methods well known in the art of pharmacy. Such methods include the step of bringing into association a PPAR agonist with liquid carriers, solid matrices, semi-solid carriers, finely divided solid carriers or combination thereof, and then, if necessary, shaping the product into the desired delivery system.

In some embodiments, a fused implant is provided herein as a bioabsorbable fused pharmaceutical implant (see, e.g., published U.S. Published Application No. 2006/0045912) for subcutaneous administration designed to slowly release a constant and effective amount of a PPAR agonist over a prolonged period. The implant may comprise a solid dispersion of a PPAR agonist uniformly dispersed in a matrix of a suitable lipoid carrier. In some embodiments, the weight ratio of the PPAR agonist to the lipoid carrier are in the approximate range of 99:1 to 80:20, respectively.

In some embodiments, a sustained release implant can be prepared as described in U.S. Pat. No. 6,203,813. The implant includes an admixture of a PPAR agonist and a pharmaceutically acceptable carrier. The admixture is uniformly compressed into a subcutaneously implantable pellet which is effective to release levels of the PPAR agonist over desired amounts of time when subcutaneously implanted in a subject as disclosed herein.

In U.S. Pat. No. 5,021,241, there is described a solid sustained-release composition in the form of a needle-like, bar-like shape which is said to consist of an active ingredient in a pharmaceutically biodegradable carrier, such as proteins in the form of collagen, gelatin, and mixtures thereof. These compositions may be used for implanting a PPAR agonist formulation as described herein. The pharmaceutically acceptable biodegradable carriers are limited to those which can be absorbed and are subject to subject to lysis by enzymes in the body. These compositions may be prepared by mixing an aqueous solution of the active ingredient with a biodegradable carrier to incorporate the active ingredient in the carrier matrix, and then drying the mixture to a shaped product having enough strength for administering to a living body.

In formulations comprising an implant, an implant can be any suitable size or shape as long as it can be practical for use in the presently disclosed methods.

Essentially any means may be used to administer a PPAR agonist to a localized region as indicated herein as long as subcutaneous adipose tissue in the localized region is exposed in situ to the PPAR agonist. In some embodiments, a formulation comprising a PPAR agonist as disclosed herein is administered within the subcutaneous space. In some embodiments, a formulation comprising a PPAR agonist as disclosed herein is administered within subcutaneous adipose tissue. In some embodiments, a formulation comprising a PPAR agonist as disclosed herein is administered within the vicinity of subcutaneous adipose tissue. In some embodiments, “within the vicinity of” may refer to a distance in the range of about 0.01 cm to about 20 cm.

In some embodiments, subcutaneous adipose tissue excludes visceral fat. In some embodiments, subcutaneous adipose tissue may include visceral fat.

In some embodiments, a composition as disclosed herein may be delivered by subcutaneous injection. A subcutaneous injection is a method of delivering formulations into, for example, the space between the skin and the muscle layer, which typically includes subcutaneous adipose tissue, using a syringe filled with the formulation, which is attached to a needle.

Needleless injection devices are disclosed in U.S. Pat. Nos. 5,938,637, 7,320,677, and 6,447,475 and may be used in some embodiments of the methods disclosed herein. Such needleless injection devices are particularly useful to deliver material to subcutaneous adipose tissue. In some embodiments, a needleless injection device may be used to propel, for example, a sustained release formulation that contains a PPAR agonist toward the surface of the individual's skin and into the subcutaneous space. The material is propelled at a sufficient velocity such that upon impact with the skin it penetrates the surface of the skin, and permeates the skin tissue.

Iontophoretic drug delivery systems are disclosed, e.g., under the trademark of IONSYS™, in U.S. Pat. No. 4,281,709. (See also, U.S. Published Pat. Application Nos. 20040267232, 20050148996 and 20070060862). Such delivery systems include a patch with a medicated surface or reservoir, and a controller, which supplies an electric current, resulting in an iontophoretic drug delivery. In some embodiments, an iontophoretic device may be used to deliver, e.g., a solution or a suspension that contains a PPAR agonist into the individual's skin.

In some embodiments, a PPAR agonist can be administered by transdermal means. For transdermal administration, penetrants appropriate to the barrier to be permeated are used in the formulation. Such penetrants are generally known in the art, and include, for example, detergents which may be used to facilitate permeation. For topical administration, the compound(s) may be formulated into ointments, salves, gels, or creams as generally known in the art. A transdermal unit dosage form can be formulated to provide a sustained release of PPAR agonist over a desired time period in the range of about one week to about ten years, such as, e.g., one week, two weeks, a month, 2 months, 4 months, 6 months, 1 year, 2 years, 5 years, 10 years or more, as indicated herein for other unit dosage forms.

For topical administration to the epidermis, a PPAR agonist may be formulated as ointments, creams or lotions, or as the active ingredient of a transdermal patch. Suitable systems are disclosed, for example, in Fisher et al. (U.S. Pat. No. 4,788,603, incorporated herein by reference) or Bawas et al. (U.S. Pat. Nos. 4,931,279, 4,668,504 and 4,713,224). Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredient may also be delivered via iontophoresis, e.g., as disclosed in U.S. Pat. No. 4,140,122, 4,383,529, or 4,051,842.

In some embodiments, an implant can be administered subcutaneously using a hollow needle implanting gun, for example of the type disclosed in U.S. Pat. No. 4,474,572. The diameter of the needle may be adjusted to correspond to the size of the implant used.

In some embodiments, a PPAR agonist is delivered by administration of a sustained release formulation into the subcutaneous space (e.g., into the subcutaneous adipose tissue). In some embodiments, sustained release is that occurring over at least one hour, at least one week, one month, one year, five years, or ten years, with longer periods of release being contemplated. In some embodiments, the sustained release occurs over the range of about one week to about twenty years. In some embodiments, release is uniform, but variations in the release profile, such as, e.g., an intermittent release profile, are acceptable. A depleted sustained formulation may be replaced as needed.

In some embodiments, a sustained release formulation is administered that releases an amount of PPAR agonist less than 10 mg, less than 5 mg, less than 3 mg, less than 1 mg, less than 0.5 mg, less than 0.1 mg, less than 10 μg, less than less than 1 μg, less than 0.01 μg, less than 0.001 μg, less than 0.0001 μg, or less than 0.00001 μg per day per subject. In some embodiments, a sustained release formulation is administered that releases an amount of PPAR agonist in the range of about 0.00001 μg per day to about 50 mg per day, in the range of about 0.00001 μg per day to about 10 mg per day, in the range of about 0.00001 μg per day to about 1 mg per day, in the range of about 0.00001 μg per day to about 100 μg per day, in the range of about 0.0001 μg per day to about 10 μg per day, in the range of about 0.001 μg per day to about 5 μg per day, in the range of about 0.001 μg per day to about 2 μg per day, or in the range of about 0.001 μg per day to about 1 μg per day. In some embodiments, a sustained release formulation is administered that releases about 1 μg per day.

In some embodiments, a sustained release formulation is administered that releases an amount of PPAR agonist in the range of about 0.00001 nmole per day to about 500 μmole per day per subject or in the range of about 0.00001 nmole per day to about 1000 nmole per day per subject. In some embodiments, a sustained release formulation is administered that releases about 0.1 nmole, about 0.5 nmole, about 1 nmole, about 2 nmole, about 4 nmole, about 5 nmole, about 8 nmole, about 9 nmole, about 10 nmole, about 15 nmole, about 20 nmole, about 50 nmole, about 100 nmole, about 200 nmole, about 500 nmole, or about 1000 nmole of a PPAR agonist per day.

In some embodiments, a sustained release formulation is administered that releases rosiglitazone maleate (MW=473.52 g/mole (357.44 g/mole free base)) in the range of about 0.00001 pg per day to about 2 mg per day, in the range of about 0.0001 μg per day to about 1000 μg per day, in the range of about 0.0001 pg per day to about 100 μg per day, in the range of about 0.001 pg per day to about 10 μg per day, or in the range of about 0.01 pg per day to about 5 μg per day. In some embodiments, a sustained release formulation is administered that releases about 0.001 μg, about 0.01 μg, about 0.1 μg, about 0.5 μg, about 1 μg, about 2 μg, about 4 μg, about 5 μg, about 6 μg, about 7 μg, about 8 μg, about 9 μg, about 10 μg, about 50 μg, about 100 μg, about 200 μg, about 500 μg, or about 1000 μg of rosiglitazone maleate per day.

In some embodiments, a sustained release formulation is administered that releases pioglitazone hydrochloride (MW=392.90 g/mole), in the range of about 0.00001 μg per day to about 10 mg per day, in the range of about 0.00001 μg per day to about 1 mg per day, in the range of about 0.00001 μg per day to about 200 μg per day, in the range of about 0.0001 μg per day to about 10 μg per day, or in the range of about 0.01 pg per day to about 5 μg per day. In some embodiments, a sustained release formulation is administered that releases about 0.001 μg, about 0.1 μg, about 0.1 μg, about 0.5 μg, about 1 μg, about 2 μg, about 4 μg, about 5 μg, about 6 μg, about 7 μg, about 8 μg, about 9 μg, about 10 μg, about 20 μg, about 50 μg, about 100 μg, about 200 μg, about 400 μg, about 800 μg, about 1000 μg, about 2000 μg, about 4000 μg, about 5000 μg, or about 10000 μg of pioglitazone hydrochloride per day.

A unit dosage form can be formulated to provide a sustained release of PPAR agonist over a time period in the range of about one day to about twenty years or more, such as, e.g., one day, a week, two weeks, a month, 2 months, 3 months, 4 months, 5 months, 6 months, 1 year, 2 years, 5 years, 10 years or more. As a non-limiting example, a unit dosage form may include sufficient PPAR agonist for release over a one month period. As a non-limiting example, if the dose is formulated to release about 1 μg of PPAR agonist per day, then the unit dosage form would contain at least about 30 μg. It will be understood that the formulation will include somewhat more than this amount of agonist (i.e., an “overage”) to compensate for short-term transients, such as initial transients, in the release rate of the agonist. In some embodiments, a unit dosage form of at least about 365 μg PPAR agonist releases about 1 μg per day over a one year period. Other unit dosage forms can be similarly formulated. In some embodiments, a unit dosage form contains about 0.001 μg, about 0.01 μg, about 0.1 μg, about 1 μg, about 2 μg, about 5 μg, about 10 μg, about 20 μg, about 30 μg, about 60 μg, about 90 μg, about 120 μg, about 150 μg, about 180 μg, about 240 μg, about 365 μg, about 480 μg, about 730 μg, about 1095 μg, about 1825 μg, about, about 10 mg, about 30 mg, about 1825 mg, about 2 g, about 5 g, about 10 g, about 20 g, about 50 g, about 100 g, or more, of a PARR agonist. In some embodiments, a unit dosage form can be formulated to provide a sustained release of PPAR agonist to subcutaneous adipose tissue in a localized area of a subject over a selected period, including days, weeks, months, years, or decades, even up to the lifetime of the subject.

A unit dosage form can be formulated to provide release of PPAR agonist over a desired time period. As a non-limiting example, a unit dosage form may include sufficient PPAR agonist for release over a one month period. For example, if the dose is formulated to release 1 pmole per kg per day, then the unit dosage form would contain 1 pmole per kg per day×30 days×70 kg, or at least about 2100 pmoles (plus overage). This calculation assumes a 70 kg body weight. Other unit dosage forms can be similarly formulated to release PPAR agonist over periods ranging from one week to ten years, or more.

In some embodiments, a unit dose is formulated based on the amount released per day (e.g., 1 μg per day) and the duration of release. A clinician may then introduce one or more of such unit doses, e.g., at different introduction sites (e.g., injection sites or implantation sites), and in one or more a selected localized areas of the body. A non-limiting example of such a unit dosage form includes a release rate of about 1 μg per day with a duration of release of 365 days; the unit dosage form would then initially contain at least about 365 μg (plus overage) of PPAR agonist. Other dosage forms may be formulated in which the rate of release can range from about 0.001 μg per day to about 1 mg, or more, per day. The duration of release can range, for example, from one week to one year, to 5 years, to 10 years, 20 years, or more. In some embodiments, a dosage form may be formulated to release any desired dose per day over the lifetime of the subject. In some embodiments, dosage forms may be formulated to release about 1 ng, about 10, ng, about 100 ng, about 1 μg, about 10 μg, about 100 μg, about 1000 μg, or more, per day, over a period ranging from one day up to the lifetime of the subject.

In some embodiments, a unit dosage form can be formulated to provide an extended release of PPAR agonist over a desired time period. As a non-limiting example, a unit dosage form may include sufficient PPAR agonist for release during a one month period. If the dose is formulated to release 1 μg per kg per day, then the unit dosage form would contain 1 μg per day (×30 days), or at least about 30 μg (plus overage). Other unit dosage forms can be similarly formulated.

Any of the doses described above can be considered as a total dose administered to a subject, wherein, in some embodiments, the total dose may be divided among multiple localized areas and/or sites within an area. In some embodiments, the number of individual doses administered ranges from 1 to 1000, or more. For example, a total dose of 4 μg could be administered as four separate 1 μg doses.

In some embodiments, plasma levels, such as peak plasma levels, or steady-state levels, of a PPAR agonist may be monitored during treatment with a PPAR agonist as disclosed herein.

In some embodiments, provided herein are methods of treating type 2 diabetes in a mammal, wherein the methods comprise administering subcutaneously to the mammal a sustained release formulation comprising a PPAR agonist, wherein the formulation releases a therapeutically effective amount of the PPAR agonist. Although one objective of the present methods is to expose subcutaneous adipose tissue to PPAR agonist, as indicated hereinabove, at least some of the PPAR agonist (and its active metabolites) will eventually distribute into the blood stream and reach a steady state concentration therein. The plasma steady state concentration of a PPAR agonist will vary depending on the release rate of the PPAR agonist from sustained release formulations as described herein.

In some embodiments, an empirical relationship between release rate and steady state plasma concentration may be determined. For example, a series of formulations having different release rates of PPAR agonist may be administered, and the resulting plasma steady state concentrations, and/or AUC₀₋₂₄ₕ, of the PPAR agonist (and its active metabolites) may be determined.

Approved oral doses of rosiglitazone (maleate) are 4 mg per day and 8 mg per day. It has been reported, in some studies, that oral administration of 8 mg rosiglitazone maleate results in a peak plasma concentration of about 461₁₄ per liter (see, e.g., www.accessdata.fda.gov/drugsatfda_docs/nda/99/21071_Avandia_biopharmr_P1.pdf). By use of the presently disclosed methods and formulations, unexpectedly low daily doses of rosiglitazone produce therapeutic results. The use of the present formulations and methods results in steady state plasma levels of rosiglitazone which are well below the plasma levels reported after oral administration, and which are low enough to substantially avoid adverse side effects of the drug. Suitable doses of a PPAR agonist, such as rosiglitazone, which are low enough to substantially avoid adverse side effects, may be determined by conventional dose/response and statistical analysis.

In some embodiments of the present methods, the PPAR agonist administered into the subcutaneous space is rosiglitazone, and the amount of the rosiglitazone released from the formulation results in a steady state plasma concentration of rosiglitazone ranging from about 0.0001 μg per liter to about 100 μg per liter, ranging from about 0.0001 μg per liter to about 50 μg per liter, ranging from about 0.001 μg per liter to about 25 μg per liter, ranging from about 0.001 μg per liter to about 10 μg per liter, or ranging from about 0.1 μg per liter to about 10 μg per liter. In some embodiments, the PPAR agonist is rosiglitazone, wherein the amount of the rosiglitazone released results in a plasma concentration of rosiglitazone at steady state which does not exceed about 0.01 μg per liter, which does not exceed about 0.1 μg per liter, which does not exceed about 1 μg per liter, which does not exceed about 5 μg per liter, which does not exceed about 10 μg per liter, which does not exceed about 25 μg per liter, which does not exceed about 50 μg per liter, which does not exceed about 75 μg per liter, which does not exceed about 100 μg per liter, which does not exceed about 200 μg per liter, which does not exceed about 300 μg per liter, or which does not exceed about 500 μg per liter.

In some embodiments of the present methods, the PPAR agonist administered into the subcutaneous space is rosiglitazone, and the amount of rosiglitazone released from the formulation results in a plasma exposure (AUC₀₋₂₄ₕ) of rosiglitazone at steady state ranging from about 0.0001 ng-h/mL to about 1000 ng-h/mL, ranging from about 0.0001 ng-h/mL to about 300 ng-h/mL, ranging from about 0.0001 ng-h/mL to about 100 ng-h/mL, ranging from about 0.001 ng-h/mL to about 25 ng-h/mL, ranging from about 0.001 ng-h/mL to about 10 ng-h/mL, or ranging from about 0.01 ng-h/mL to about 1 ng-h/mL. In some embodiments, the PPAR agonist is rosiglitazone, wherein the amount of rosiglitazone released results in a plasma exposure (AUC₀₋₂₄ₕ) of rosiglitazone at steady state, which does not exceed about 0.01 ng-h/mL, which does not exceed about 0.1 ng-h/mL, which does not exceed about 1 ng-h/mL, which does not exceed about 5 ng-h/mL per liter, which does not exceed about 10 ng-h/mL, which does not exceed about 25 ng-h/mL, which does not exceed about 50 ng-h/mL, which does not exceed about 75 ng-h/mL, which does not exceed about 100 ng-h/mL, which does not exceed about 200 ng-h/mL, which does not exceed about 300 ng-h/mL, which does not exceed about 500 ng-h/mL, or which does not exceed about 1000 ng-h/mL.

Quantification of rosiglitazone may be carried out by chromatographic and mass spectral techniques known to one of skill in the art (see, e.g., Kim et al. (2009) Journal of Chromatography B 877:1951-1956).

“AUC₀₋₂₄ₕ” as used herein, means area under the plasma concentration-time curve, as calculated by the trapezoidal rule, over a 24-hour interval. Plasma AUC₀₋₂₄ₕ is one measure of systemic exposure. In some embodiments, other parameters may also be used, including Cmax, Css, etc. As used herein, the term “Cmax” is the maximum plasma concentration obtained during a dosing interval. “Css” is the steady state concentration.

As used herein, the term “plasma concentration at steady state” is the concentration reached after administration of a sustained release formulation of a PPAR agonist as disclosed herein. Once steady state is reached, there may be minor peaks and troughs in the plasma concentration of the PPAR agonist, but the concentration will remain substantially constant until the formulation approaches depletion.

Approved oral doses of pioglitazone (HCl) are 30 and 45 mg per day. It has been reported that oral administration of 30 mg pioglitazone results in a peak plasma concentration of pioglitazone and its active metabolites of about 146 μg per liter (see, e.g., www.accessdata.fda.gov/drugsatfda_docs/nda/99/021073A_Actos_clinphrmr_P1.pdf).

By use of the disclosed methods and formulations, unexpectedly low doses of pioglitazone produce therapeutic results. The use of these low doses results in low steady-state plasma levels of pioglitazone and its active metabolites, well below the plasma levels reported after oral administration, and thereby substantially avoids adverse side effects of the drug.

In some embodiments of the present methods, the PPAR agonist administered subcutaneously is pioglitazone, and the amount of the pioglitazone released from the formulation results in a steady state plasma concentration of pioglitazone and its active metabolites ranging from about 0.0001 μg per liter to about 500 μg per liter, ranging from about 0.01 μg per liter to about 100 μg per liter, ranging from about 0.1 μg per liter to about 50 μg per liter, or ranging from about 1 μg per liter to about 25 μg per liter. In some embodiments, the PPAR agonist is pioglitazone, wherein the amount of the pioglitazone released results in a plasma concentration of pioglitazone and its active metabolites at steady state which does not exceed about 0.01 μg per liter, which does not exceed about 0.1 μg per liter, which does not exceed about 1 μg per liter, which does not exceed about 10 μg per liter, which does not exceed about 25₁₄ per liter, which does not exceed about 50 μg per liter, which does not exceed about 75 μg per liter, which does not exceed about 100 μg per liter, which does not exceed about 150 μg per liter, which does not exceed about 200 μg per liter, which does not exceed about 300 μg per liter, which does not exceed about 400 μg per liter, or which does not exceed about 500 μg per liter.

In some embodiments of the present methods, the PPAR agonist administered subcutaneously is pioglitazone, and the amount of poiglitazone released from the formulation results in a plasma exposure (AUC₀₋₂₄ₕ) of poiglitazone and its active metabolites ranging from about 0.0001 μg-h/mL to about 50 μg-h/mL, ranging from about 0.0001 μg-h/mL to about 20 μg-h/mL, ranging from about 0.001 μg-h/mL to about 10 μg-h/mL, ranging from about 0.001 μg-h/mL to about 2.5 μg-h/mL, or ranging from about 0.001 μg-h/mL to about 5 μg-h/mL. In some embodiments, the PPAR agonist administered subcutaneously is poiglitazone, wherein the amount of poiglitazone released from the formulation results in a plasma exposure (AUC₀₋₂₄ₕ) of poiglitazone and its active metabolites at steady state which does not exceed about 0.00001 μg-h/mL, which does not exceed about 0.0001 μg-h/mL, which does not exceed about 0.001 μg-h/mL, which does not exceed about 0.01 μg-h/mL per liter, which does not exceed about 0.05 μg-h/mL, which does not exceed about 0.1 μg-h/mL, which does not exceed about 0.2 μg-h/mL, which does not exceed about 0.5 μg-h/mL, which does not exceed about 1.5 μg-h/mL, which does not exceed about 2.5 μg-h/mL, or which does not exceed about 5 μg-h/mL.

Quantification of pioglitazone and its active metabolites may be carried out by chromatographic and mass spectral techniques known to one of skill in the art (see, e.g., Zhong et al. (1996) J. Pharm. Biomed. Anal. 14:465-473; Sengupta et al. (2010) Biomed. Chromatogr. 24:1342-1349; Tanis et al. (1996) J. Med. Chem. 39:5053-5063).

It will be appreciated that a sustained release dosage form, once administered, may release a PPAR agonist at a somewhat higher rate initially (i.e., transiently), and thereafter at a substantially constant rate. A dosage will typically include more PPAR agonist than a simple calculation would indicate in order to compensate for these transients. As a non-limiting example, a unit dosage that releases 1 μg per day, as illustrated above, will contain at least about 365 μg, and may actually contain about 370 pg to about 400 pg. Suitable formulations can be designed using routine experimentation and empirical observation.

It will also be appreciated that a PPAR agonist administered in a sustained release formulation, as disclosed herein, will alter the subcutaneous adipose tissue over time and will therefore require a period of time, such as, e.g., days, weeks, or months, to achieve a therapeutic effect. As a non-limiting example, a formulation, once administered to a subject having hyperglycemia may require a period of 1 to 6 months to achieve euglycemia. In the interim, however, the PPAR agonist treatment will have some beneficial effects, such as lowering the severity of the hyperglycemia.

After administration, when a dosage form of PPAR agonist nears depletion, the release rate may not be constant, and may decrease. A new dose may be administered in order to maintain a consistent exposure of the subcutaneous adipose tissue to the PPAR agonist. The timing of administration of the new dose may be calculated based on the expected lifetime of the initial dose, and/or may be based on a measurement such as levels of PPAR agonist measured in the blood stream.

Some embodiments of effective daily doses of PPAR agonist are described above. In some embodiments, the present formulations and doses assume a body weight of a person in the range of 10 kg to 200 kg. In some embodiments, the present formulations and doses assume a 20 kg body weight, a 50 kg body weight, or a 70 kg body weight for a person. The exact dose to be administered may be determined by the attending clinician and may be further dependent upon the efficacy of the particular PPAR agonist used, as well as upon the age, weight and condition of the subject.

In some embodiments, unit doses are administered to more than one localized area, and may be administered to more than one site within each localized area.

In some embodiments, the present methods concern administering a sustained release PPAR agonist formulation subcutaneously within, or in the vicinity of, subcutaneous adipose tissue in a localized area of the body of a subject. In some embodiments, a PPAR agonist is administered to one or more localized areas of the body, and can be administered at one or more sites within each localized area. In some embodiments, the localized area can be selected from abdomen, chest, breast, flank, inguinal region, back, trunk, hip, suprascapular region, leg, arm, thigh, buttock, and combinations thereof.

In some embodiments, a subject gains weights due to an increase in subcutaneous adipose tissue mass (and supporting tissue) due to the administration of a PPAR agonist as described herein.

In some embodiments, the administration of a PPAR agonist formulation can be used, for example, to distribute any resulting accumulation of subcutaneous fat at different regions of the body of the subject. In some embodiments, the administration may be performed in any desired pattern over the body of the subject, and can be, e.g., asymmetrically administered, or substantially symmetrically administered. In some embodiments, the administration may be performed on each side of the body, such as, e.g., symmetrically administered to both right and left regions of the body. For example, the PPAR agonist formulation can be administered to the right and left buttock, right and left leg, right and left hip, right and left regions of the back, right and left hip, and/or right and left suprascapular region, etc. In some embodiments, the PPAR agonist formulation can be distributed substantially symmetrically to left and right regions and/or to anterior and posterior regions of the body.

A sustained release unit dosage form, once administered subcutaneously within or near adipose tissue in a localized area of a body, will slowly release PPAR agonist. In some embodiments, the localized area can be subjected to periodic gentle massage and/or warming, such as once a day, in order to facilitate distribution of the PPAR agonist throughout the subcutaneous adipose tissue of the localized area.

By administering a PPAR agonist at a plurality of regions and/or sites within each region, and by other methods as indicated above, potential uneven accumulation of adipose, such as bumps or other cosmetically undesired accumulation, may be substantially avoided.

In some embodiments, a subject gains weights due to an increase in subcutaneous adipose tissue mass (and supporting tissue) due to the administration of a PPAR agonist as described herein, but because the subcutaneous adipose tissue exhibits a higher metabolic activity due to the presently described treatment methods, a decrease in adipose tissue mass at other locations may occur. For example, visceral adipose mass may decrease as a result of use of the present methods. In some embodiments, treatment with a PPAR agonist as described herein may result in redistribution of adipose tissue or lipids from the visceral or omental fat depot, bone marrow, liver, heart, pancreas, gonads (e.g., ovaries) or other organs to subcutaneous adipose tissue or to the subcutaneous space. Such redistribution of fat from critical organs to subcutaneous adipose tissue will exert beneficial effects in type 2 diabetes, non-alcoholic fatty liver disease, improved fertility, decreased osteoporosis, etc. Additionally, in some embodiments, the redistribution of lipids may substantially offset weight gain from the increase in mass of the subcutaneous adipose tissue.

The optimal mode of administration of a PPAR agonist to a subject as disclosed herein depends on factors known in the art such as the particular disease or disorder, the desired effect, and the type of subject. While the present methods and compounds will typically be used to treat human subjects, they may also be used to treat similar or identical diseases in other vertebrates such as other primates, farm animals such as swine, cattle and poultry, and sports animals and pets such as horses, dogs and cats.

Where desired, a PPAR agonist as disclosed herein may be used in combination with one or more other types of therapeutic agents including antidiabetic agents, anti-obesity agents, antihypertensive agents, platelet aggregation inhibitors, and/or anti-osteoporosis agents, which may be administered in the same dosage form. Other examples of agents (see, e.g., Flynn et al. (2008)) include: tumor necrosis factor alpha (TNFα), growth hormone, epidermal growth factor, insulin, triiodothyronine, glucocorticoids, biotin, pantothenate, isobutylmethylxanthine, insulin-like growth factor 1 (IGF-1), and basic fibroblast growth factor.

The present disclosure also provides pharmaceutical kits or packs useful, for example, for the treatment of type 2 diabetes and related disorders, which comprise one or more containers containing a pharmaceutical composition comprising a therapeutically effective amount of a compound of a PPAR agonist, e.g., one or more unit doses, with or without additional active agents. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

Measurement of Anthropometric and Biochemical Parameters

In some embodiments, various parameters of the subject and of the targeted subcutaneous adipose tissue may be analyze using assays known in the art in order to determine the effectiveness of PPAR agonist administration as described herein.

In some embodiments of the present methods, the increase in size or mass of subcutaneous adipose tissue in a localized area exposed to PPAR agonist as disclosed herein results in weight gain in the subject. The weight gain can occur over some or all of the treatment period, such as a period of weeks, months, years, or longer. The weight gain will vary depending on the amount, duration, and rate of delivery of administration of the PPAR agonist to the subject. The administration of PPAR agonist can be varied by the number of localized areas administered, and by the number of introduction sites per localized area. The total weight gain, and location of weight gain, can be modulated by the number and location of localized areas to which the formulation is administered, and by the number of sites of introduction per localized area. In some embodiments, the weight gain can be in the range of about 0.5 gram to about 30 kg, about 10 gram to about 20 kg, about 0.5 kg to about 10 kg, about 1 kg to about 5 kg, about 1 kg to about 4 kg, or about 1 kg to about 3 kg. In some embodiments, the weight gain is about 0.1 kg to about 30 kg. In some embodiments, the total dose of PPAR agonist can be divided and administered in a plurality of locations, as described above. In some embodiments, the administration may be made to both the right and left sides of the body, such that any resulting increase in adipose tissue is substantially symmetrically distributed over the body (e.g., from the right side to left side, and/or from anterior to posterior).

In some embodiments, adipose tissue from a localized area can be sampled (e.g., by use of biopsy or liposuction) for analysis. In some embodiments, concentration of the drug in subcutaneous adipose tissue can be analyzed in situ (e.g., via biopsy or microdialysis). Morphological and metabolic parameters of subcutaneous adipocytes may be monitored before and during exposure to a PPAR agonist as described herein.

Adipocytes of untreated subcutaneous adipose tissue typically have a single vacuole (unilocular) and have few mitochondria, whereas adipocytes of PPAR agonist exposed subcutaneous adipose tissue have a morphology that is multi-vacuolar (multilocular), and have many mitochondria. In some embodiments, the changes to adipocytes in the subcutaneous adipose tissue of a subject are monitored by microscopically determining the presence and quantity of multilocular adipocytes. In some embodiments, the number of vacuoles per adipocyte in the subcutaneous adipose tissue increases during the exposure to a PPAR agonist as disclosed herein. In some embodiments, the number of vacuoles per adipocyte increases in the range of about 2-fold to about 100-fold or more.

White adipocytes typically are characterized by being unilocular (i.e., possessing a single fat vacuole). In some embodiments, the vacuole size of adipocytes in subcutaneous adipose tissue exposed to PPAR agonist decreases. In some embodiments, the vacuole size can decrease by 10%, 20%, 30%, 50%, 70%, 90%, or more. In some embodiments, the number of adipose cells per unit volume in the subcutaneous adipose tissue increases during exposure to a PPAR agonist. In some embodiments, the number of adipose cells per ml volume increases in the range of about 10% to about 1000%. In some embodiments the number of adipose cells per ml volume increases by about 30% and about 80%. In some embodiments, the number of small adipocytes increases, and the ratio of small to large adipocytes increases. The percent of large vs small adipocytes, and the number of cells per unit volume, may be estimated by, e.g., H&E staining of adipose tissue, flow cytometry and/or microscopic analysis.

The levels of one or more markers (e.g., protein and/or mRNA) indicative of differentiated subcutaneous adipose tissue may be determined. For example, uncoupling protein-1 (UCP1) is a proton transporter that allows protons to leak across the mitochondrial inner membrane, thereby dissipating the electrochemical gradient normally used for ATP synthesis. In some embodiments, subcutaneous adipose tissue is analyzed for UCP1 peptide or mRNA to monitor the effect of the present methods.

In some embodiments, the effect of exposure to PPAR agonists as disclosed on subcutaneous adipose tissue may be monitored by measuring the expression of a mitochondrial gene, non-limiting examples of which include cytochrome C, cox4i1, coxIII, cox5b, atpase b2, cox II, atp5o, and ndufb5.

Adipocytes exposed to a PPAR agonist as disclosed herein are characterized by densely packed mitochondria that contain UCP1 in their inner mitochondrial membrane. In some embodiments, subcutaneous adipose tissue can be analyzed by electron microscopy to determine the elevation of mitochondrial volume density. In using electron microscopy to calculate mitochondrial volume density, a grid may be laid on randomly selected micrographs (e.g., n>20), and the number of points falling onto mitochondria is expressed as a fraction of those landing on a cell area. In some embodiments, subcutaneous adipocytes treated as described herein reveal at least a 2-fold increase, at least a 10-fold increase, or at least a 100-fold increase in mitochondrial volume density.

Other mitochondrial parameters may be determined (such as, e.g., described in the Examples herein and in U.S. Pat. No. 6,140,067) and may include mitochondrial enzymes and ATP biosynthesis factors, mitochondrial mass, mitochondrial number, mitochondrial DNA content, cellular responses to elevated intracellular calcium, apoptogenesis, free radical production, and the like.

Adipocyte respiration may be total or uncoupled respiration and may be measured by oxygen consumption.

In some embodiments, after subcutaneous administration of a sustained release formulation comprising a PPAR agonist as disclosed herein within a localized region, the concentration of the PPAR agonist within the subcutaneous space, and/or the subcutaneous adipose tissue within the localized region may be determined. The concentration of the agonist at various sites within the localized region, or an average of the agonist concentrations from said sites, may be determined. The subcutaneous concentration of PPAR agonist can be determined by any suitable method (see, e.g., Chaurasia et al (2007) Pharmaceutical Res. 24:1014-1025 and Stahle et al. (1991) Life Sci. 49:1853-1858; www.ashp.org/DocLibrary/Bookstore/P2418-Chapter1.aspx; de Lange et al. (1997) Brain Res. Rev. 25:27-49; Komoroski (1994) Anal. Chem. 66:1024A-1033A; Lovich et al. (1999) PNAS 96:11111-11116) which may include microdialysis, NMR, PET, or other methods. Levels of a PPAR agonist can be determined in situ, or can be determined in adipose tissue samples obtained by biopsy or liposuction. In animal model systems, subcutaneous adipose tissue may be removed and analyzed after necroscopy. In some embodiments, a PPAR agonist may be radio-labeled, and the concentration (i.e., the amount per unit volume) of the agonist in the plasma, in the subcutaneous adipose tissue, and/or in the subcutaneous space may be determined over a course of time. In some embodiments, the ratio of a PPAR agonist concentration in the subcutaneous space (or in the subcutaneous adipose tissue) to the steady-state plasma concentration of the PPAR agonist (or of the agonist plus its active metabolites) may be determined. In some embodiments, after administration of a formulation as disclosed herein, the ratio of a PPAR agonist concentration in the subcutaneous adipose tissue to the steady-state concentration of the agonist in plasma is in the range of about 2 to about 10000, in the range of about 5 to about 1000, or in the range of about 10 to about 100. In some embodiments, the ratio is at least about 5, at least about 10, at least about 50, at least about 100, at least about 500, at least about 1000, at least about 10000 or more.

Pharmaceutical compositions and methods disclosed herein provide a sustained blood sugar lowering action, blood lipid lowering action, insulin sensitizing action, and/or blood insulin lowering action. In some embodiments, parameters such as the following are measured in plasma and/or serum: glucose levels; fasting glucose levels; insulin; lipid composition; cholesterol and triglyceride levels.

The blood sugar lowering action of a formulation as disclosed herein can be evaluated by comparing the concentration of glucose or Hb (hemoglobin) A_{1c} in venous blood plasma before administration and after administration of the formulation. HbA_{1c} (i.e., glycosylated hemoglobin) is an important index of blood sugar control which is not easily influenced by rapid blood sugar changes in diabetic subjects.

In some embodiments, the methods and compositions disclosed herein can be used in the treatment of one or more of glucose intolerance, hyperinsulinemia, insulin resistance and hyperlipidemia. Glucose intolerance is characterized by a pathological state in which the fasting plasma glucose level is less than 140 mg per deciliter and the 30-, 60-, or 90-minute plasma glucose concentration following a glucose tolerance test exceeds 200 mg per deciliter.

Hyperinsulinemia is a condition in which the level of insulin in the blood is higher than normal. Hyperinsulinemia is caused by overproduction of insulin by the body and is related to insulin resistance.

Plasma glucose concentrations may be measured by glucose oxidase method (YSI glucose analyzer, Yellow Springs Instrument, Yellow Springs, Ohio, USA). Plasma total cholesterol, triglyceride and HDL-cholesterol may be determined by enzymatic methods, e.g., using a Hitachi 7150 autochemistry analyzer. Nonesterified fatty acid (NEFA) may be measured by enzymatic methods (e.g., NEFAzyme kit, Eiken, Japan). Serum may be measured by radioimmunoassay insulin (e.g., Diagnostic Products Co, USA).

Insulin resistance occurs when the body does not respond to the insulin made by the pancreas, and glucose is less able to enter the cells. Subjects with insulin resistance may or may not go on to develop type 2 diabetes. Any of a variety of tests in current use can be used to determine insulin resistance, including: the Oral Glucose Tolerance Test (OGTT), Fasting Blood Glucose (FBG), Normal Glucose Tolerance (NGT), Impaired Glucose Tolerance (IGT), Impaired Fasting Glucose (IFG), Homeostasis Model Assessment (HOMA), the Quantitative Insulin Sensitivity Check Index (QUICKI) and the Intravenous Insulin Tolerance Test (IVITT). See also, De Vegt (1998) “The 1997 American Diabetes Association criteria versus the 1985 World Health Organization criteria for the diagnosis of abnormal glucose tolerance: poor agreement in the Hoorn Study.” Diab. Care 21:1686-1690; Matthews (1985) “Homeostasis model assessment: insulin resistance and B-cell function from fasting plasma glucose and insulin concentrations in man.” Diabetologia 28:412-419; Katz (2000) “Quantitative Insulin Sensitivity Check Index: A Simple, Accurate Method for Assessing Insulin Sensitivity In Humans.” JCE & M 85:2402-2410.

Blood pressure, height, weight, and circumferences of waist and hip of the subject receiving treatment by the disclosed methods may be measured by conventional methods. Total body fat content, expressed as fat mass (kg) may be determined using bioelectric impedance analyzer (e.g., Inbody 2.0, Biospace CO., Ltd.). Percent body fat (%) may be calculated using the following formula: fat mass (kg) divided by body weight (kg)×100. BMI may be determined using known methods. Subcutaneous and visceral fat may be analyzed by known methods (e.g., using PET/CT, ultrasound, and/or MRI, skin flap thickness, biopsy, or other means).

In some embodiments, pharmaceutical compositions and methods disclosed herein are useful for preventing or treating type 2 diabetes and related conditions, substantially without apparent detection of adverse side effects. As noted above, adverse side effects may include myocardial infarction, stroke, macular edema, pulmonary edema, peripheral edema, fluid retention, cardiac hypertrophy, cardiovascular disease, atheroscloerosis, congestive heart failure, bone fracture, osteoporosis, adipogenesis in bone marrow, bladder cancer, and hepatitis. Conventional methods may be used to test for these conditions in animal model systems, and in human subjects where feasible, and statistical tests performed to compare treatment groups with controls. For example, such methods may include analysis by histopathology, light microscopy, electron microscopy, micro CT bone density scan, bone mineral detection, bone compression, three-point bending test, echocardiogram, histomorphometry, tumor marker analysis, urinary bladder nodule detection, transitional cell tumor detection, and hepatotoxicity marker (e.g., alanine transferase, alkaline phosphatase, bilirubin, etc.) assay. Non-limiting examples of urinary markers for bladder cancer include: BTA Stat; BTA Trak; NMP 22; Bladder Chek; Immunocyt; UroVysion; Cytokeratins 8, 18, 19; Telomerase-TRAP, hTert, hTR [human telomerase (hTR); human telomerase reverse transcriptase (hTert); telomeric repeat amplification protocol (TRAP)]; BLCA-4; Survivin-protein and mRNA; Microsatellite markers; Hyaluronic acid/hyaluronidase; DD23 monoclonal antibody; Fibronectin; HCG-protein and mRNA [Human chorionic gonadotropin (HCG)]; DNA promotor regions of hypermethylated tumor suppressor and apoptosis genes; and Proteomic profiles (Mass spectrometry). Non-limiting examples of markers for cardiovascular diseases include: Low-density lipoprotein; Lipoprotein(a); Apolipoprotein A1; Apolipoprotein Bho; Higher fibrinogen and PAI-1 blood concentrations; Elevated homocysteine, or even upper half of normal; Elevated blood levels of asymmetric dimethylarginine; Inflammation as measured by C-reactive protein; Elevated blood levels of brain natriuretic peptide (also known as B-type) (BNP).

Utility

The disclosed methods and compositions are of use in the treatment of type 2 diabetes. The disclosed methods and compositions are also indicated to be of use for the treatment, partial treatment, and/or prophylaxis of other diseases including insulin resistance, hyperlipidemia, hypertension, cardiovascular disease, and atherosclerosis.

In some embodiments, the disclosed methods and compositions can be used for preventing or treating diabetic complications (e.g., neuropathy, nephropathy, retinopathy, macroangiopahty, osteopenia, etc.) and can also be also used for preventing or treating diseases such as hyperlipemia, hyperinsulinemia, obesity, hyperphagia, hypertension, cardiovascular diseases (e.g., atherosclerosis, etc.), polycystic ovarian syndrome, gestational diabetes, pancreatitis, glomerulonephritis, glomerular sclerosis, hypertensive nephrosclerosis, etc., or syndromes (e.g., Syndrome X, visceral fat obesity syndrome, etc.) having some of these diseases in combination.

In some embodiments, the present methods and compositions can be used for lowering plasma levels of triglycerides and apolipoprotein B, decreasing the proportion of small, dense low-density lipoprotein (LDL) particles, decreasing total cholesterol, increasing HDL-cholesterol, lowering insulin resistance, and lowering hyperinsulinemia.

In some embodiments, by use of the present methods and formulations, a subject is able to achieve an average preprandial plasma glucose concentration in the range of 90-130 mg/dL, average bedtime plasma glucose values between 110-150 mg/dL and HbA_{1c} values less than 7%.

In some embodiments, by use of the present methods and formulations, a subject is able to achieve an average fasting blood glucose level of about 70 mg/dL to about 99 mg/dL, and a posprandial blood glucose level of about 70 mg/dL to about 140 mg/dL. In some embodiments, a subject is able to achieve an HbA_{1c} value of between about 4.5 to about 5.5.

In some embodiments, by use of the present methods and formulations, a subject is able to achieve an average fasting blood glucose level of about 72 mg/dL to about 108 mg/dL, and a postprandial blood glucose level of about 110 mg/dL to about 150 mg/dL. In some embodiments, a subject is able to achieve a 2-hour postprandial blood glucose in the range of about 90 mg per deciliter to about 144 mg per deciliter.

The American Heart Association has created a set of guidelines for triglyceride levels in the blood. Normal levels of triglyceride concentration in the blood are below 150 mg/dL. Borderline high levels are considered between 150 and 200 mg/dL and high levels between 200 and 500 mg/dL. Any concentration levels above 500 are considered seriously high risk levels. In some embodiments, by use of the present methods and formulations, a subject is able to achieve blood triglyceride levels below 150 mg/dL.

HDL levels below 40 mg/dL result in an increased risk of coronary artery disease, even in people whose total cholesterol and LDL cholesterol levels are normal. HDL levels between 40 and 60 mg/dL are considered “normal.” However, HDL levels greater than 60 mg/dL may actually protect people from heart disease. In some embodiments, by use of the present methods and formulations, a subject is able to achieve blood triglyceride levels between 40 and 60 mg/dL, and in some cases, greater than 60 mg/dL.

In some embodiments, treating a subject using the methods and formulations described herein results in euglycemia or improved euglycemic control, thus delaying the need for other therapy. In some embodiments, the treatment results in a reduction in blood triglycerides and other lipids. In some embodiments, the treatment results in a reduction in the amount of insulin that the subject requires on a daily basis to maintain euglycemia.

In some embodiments, a composition for subcutaneous administration, as described herein, is formulated to release a PPAR agonist in a daily dose that is a fraction of an effective oral daily dose of said agonist, and wherein said daily dose from subcutaneous administration of said composition provides a therapeutic effect substantially equivalent to that of said oral daily dose. Examples of such an equivalent therapeutic effect include glycemic control, reduction in the amount of insulin required, improved lipid profile, improved HDL/LDL ratios, reduction in blood triglycerides, or treatment and/or prophylaxis of other diseases indicated herein. In some embodiments, said fraction is in the range of about one half to about 1/100000^{th}. In some embodiments, said fraction is in the range of about ⅕^{th} to about 1/100000^{th}. In some embodiments, said fraction is no greater than about ⅕^{th}, 1/10^{th}, 1/100^{th}, or 1/1000^{th}.

The disclosed methods and composition provide a number of potential advantages, including: providing improved control of blood glucose which substantially avoids the often noted risk of hypoglycemia that is associated with treatment with insulin; improving compliance by eliminating daily oral dosing of the PPAR agonist; substantially eliminating excursions of lower and higher glucose often noted with lack of compliance; and substantially eliminating the need for other therapies.

“Therapeutically effective amount” means that amount of a drug or pharmaceutical agent that will elicit the biological or medical response of a tissue, system or animal that is being sought by a researcher or clinician.

In some embodiments, PPAR agonists are administered to diabetics or to non-diabetics by the disclosed methods in the treatment of conditions such as obesity, abdominal obesity, lipid disorders, dyslipidemia, hyperlipidemia, hypertriglyceridemia, hypercholesterolemia, low HDL levels and/or high LDL levels, atherosclerosis and its sequelae, carotid and coronary atherosclerosis, fluid retention, vascular restenosis, pancreatitis, neurodegenerative disease, retinopathy, nephropathy, neuropathy, gastrointestinal motility disorders, fertility and reproductive disorders.

The following examples are put forth so as to provide those of ordinary skill in the art with a complete disclosure and description of how to make and use the presently disclosed compositions and methods, and are not intended to limit the scope of what the Applicant regards as the invention nor are they intended to represent that the experiments below are all or the only experiments performed.

### Example 1

### Sustained Release Formulations

Five different sustained release formulations were prepared as shown in Table 1.

TABLE 1

| | Ingredients | Percentage % (w/w) |
|---|---|---|
| Formulation 1 | Rosiglitazone Maleate | 1.5 |
| | PLGA | 9.85 |
| | Triacetin | 88.65 |
| Formulation 2 | Rosiglitazone Maleate | 1.5 |
| | PLGA | 19.7 |
| | Triacetin | 78.8 |
| Formulation 3 | Rosiglitazone Maleate | 1.5 |
| | PLGA | 29.55 |
| | Triacetin | 68.95 |
| Formulation 4 | Rosiglitazone Maleate | 1.5 |
| | PLGA | 19.7 |
| | N-methyl Pyrrolidone (NMP) | 78.8 |
| Formulation 5 | Rosiglitazone Maleate | 1.5 |
| | PLGA | 39.4 |
| | NMP | 59.1 |

Rosiglitazone maleate particles were dissolved in pre-made PLGA:NMP (40:60) gel. Poly lactic-co-glycolic acid (PLGA), NMP and Triacetin were obtained from Sigma-Aldrich.

In vitro evaluation of the elution rate: To study the in vitro rosiglitazone release profile, the sustained release formulations that have different release rates are placed in the vials containing 15 ml of phosphate buffer saline (pH 7.4) and kept on a shaker water bath set at 37° C. and 60 oscillations per minute. Clear 1 ml samples are withdrawn at predetermined intervals during the study period. After each sampling, an equal volume of fresh buffer solution, at the same temperature, is replaced. The amount of drug released is determined using HPLC and/or mass spectral analysis.

### Example 2

### Evaluation of Sustained Release Formulations Using In Vivo Experimental Models

The efficacy of the formulations are evaluated by in vivo experimental hyperlipidemic and diabetic models of mice. Short term studies using C57Bl/6 mice: C57Bl/6 mice are used to test the biological effect of rosiglitazone given as a sustained release formulation for a period of 10 days. The formulation is injected either in the fat depots on the back or in the inguinal region of the treated group while the control group is given the polymer alone. Three different release rates of the formulation (10 μg, 100 μg, 1000 μg) per day are tested. Other release rates (such as, e.g., 0.000001 μg, 0.00001 μg, 0.0001 μg, 0.001 μg, 0.01 μg, 0.1 μg, and 1.0 μg per day) are similarly tested.

Body weight, clinical observations, and plasma glucose, insulin and lipid concentrations are monitored prior to and at various phases of the treatment. At the end of the dosing period, the animals are necropsied, and the total weight of the animals and wet weight of the adipose tissue depots are determined. A part of the adipose tissue is fixed for performing immunochemical staining and whole-mounted adipose tissues are co-immunostained for perilipin (a membrane protein that surrounds lipid droplets and is selectively expressed in adipocytes and steroidogenic cells) (Londos et al. (2005) Biochimie 87:45-9; Koh et al., (2007) J. Clin. Invest. 117:3684-95) and collagen IV (the matrix protein that surrounds each adipocyte individually as a basement membrane) (Londos et al., 2005). To determine mitochondrial biogenesis, whole-mounted adipose tissues are co-stained Mitotracker (fluorescent dye for selective binding to intracellular mitochondria), and immunostained for collagen IV or UCP-1. These double and triple co-stainings will enable visualization of mitochondrial content and UCP-1 expression in the collagen IV-covered adipocytes. Quantitative Real-time PCR is performed to characterize white adipocyte population and the brown adipocyte population by quantifying the mRNA levels of the white adipocyte specific markers—Igfbp3, DPT, Tcf21, Hoxc9 and of brown adipocyte specific markers—miR-206, myogenin, CPT-1M, PRDM16, PGC1-α and UCP-1. The expansion of the white adipocyte population is further confirmed by the expression of markers specific for a brown adipocyte-like population such as UCP-1 and voltage dependent anion carrier (VDAC), and compared to control groups that are not treated with the drug. Adipose tissue is further examined by light microscopy, following H&E staining, to determine the number of vacuoles, size of the vacuoles, and number of adipocytes per field, to determine the morphological changes.

Long Term Studies:

Additional, long term studies in rodent models of diabetes are conducted to assess the effectiveness of the treatments. The db/db mouse model (8-9 week old) are used for evaluating the effect of the administration of sustained release formulations of rosiglitazone maleate in decreasing hyperinsulinemia, hyperlipidemia and glucose intolerance apart from the expansion of adipose tissue. A formulation of rosiglitazone that is optimized to release rosiglitazone over a 3-month period is administered subcutaneously and in the vicinity of subcutaneous adipose tissue. Following treatment, blood is drawn periodically to measure plasma glucose, insulin and lipids. A glucose tolerance test is also performed. At the termination of the experiment, the effect of rosiglitazone on the adipose tissue expansion and its ability to control diabetes is assessed as described above.

In Vivo Evaluation:

Animals: Animals are housed individually under standard conditions at 25° C. with a 14:10-h light-dark cycle, with free access to food and water. C57Bl/6J mice that are 6 week old and 4 week old Lepr^{db} and Lepr^{db/″} or Lep⁺ mice are obtained from the Jackson Laboratory (Bar Harbor, Me., USA). Mice are placed on either standard chow (6% fat wt/wt, RD8664; Harlan Teklad) or high-fat diet (HFD; 42% fat wt/wt, TD88137; Harlan Teklad).

(1) C57Bl/6 mice: Control and drug treated C57Bl/6 mice (n=5), 8-9 weeks old are treated for 3 weeks. The control group is administered vehicle alone and the treated groups are administered a sustained release formulation which releases PPAR agonist at different release rates, release rate 1 (RR1), release rate 2 (RR2) and release rate 3 (RR3). The positive control group mice (n=5) is given the drug at 15/mg/kg body weight in normal diet ad libitum (Table 3).

(2) Lepr^{db} (db/db) mice: Control mice and drug treated Lepr^{db} and Lepr⁺ mice (n=10) 8 week old are treated for 4 weeks and 3 months. The control group is administered vehicle alone and the treated groups are administered a sustained release formulation which releases PPAR agonist at two different release rates RR1 and RR2. The positive control group mice (n=5) are given the drug at 15/mg/kg body weight in normal diet ad libitum. The group containing Lean mice (n=5) given the implant alone serves as negative control group (Table 4).

TABLE 3

| Group | No. of animals |
|---|---|
| Control C57Bl/6 (Vehicle) | 5 |
| Treated C57Bl/6 (RR1) | 5 |
| Treated C57Bl/6 (RR2) | 5 |
| Treated C57Bl/6 (RR3) | 5 |
| Treated C57Bl/6 (Oral) | 5 |

TABLE 4

| Group | No. of animals |
|---|---|
| Control Lean (Vehicle) | 5 |
| Control db/db (Vehicle) | 10 |
| Treated db/db (RR1) | 10 |
| Treated db/db (RR2) | 10 |
| Treated db/db (Oral) | 5 |

Body weight and food consumption: The body weight and food consumption of the animals are monitored regularly in the control and treated mice.

Biochemical Parameters:

Plasma insulin, lipids and glucose: Plasma insulin is determined using commercial kits (Crystal Chem Inc). Glucose (nonfasting) is measured using a Glucometer (OneTouch Glucometer from Lifescan). Triglycerides (T2449; Sigma, USA) and FFA (700310, Cayman Chemical Company) are measured according to the manufacturer's protocol. Glucose tolerance test: 6-h fasted anesthetized mice are given 150 mg glucose by gavage through a gastric tube (outer diameter 1.2 mm), inserted in the stomach. Blood samples are taken at 0, 15, 30, 60, 90, and 120 min after glucose administration and blood glucose levels determined.

Insulin tolerance test: The test is performed on random-fed mice. The mice are injected with insulin (0.75 U/kg) (Humulin-R 100 U/ml from Ely Lilly) in ˜0.1 ml 0.9% NaCl intraperitoneally. A drop of blood (5 μl) is taken from the cut tail vein before the injection of insulin and after 15, 30, 45, and 60 min for the determination of blood glucose with a glucometer (OneTouch Ultra from Lifescan).

Adipose Tissue Analysis:

At the end of the treatment, the mice are necropsied and the subcutaneous adipose tissue pads are collected and the following parameters are determined.

(a) Wet weight

(b) Quantitating different adipocyte populations

(c) Quantitative Real-time PCR: For determination of mRNA levels, 1 μg of RNA isolated by Trizol extraction is reverse-transcribed with a High Capacity cDNA kit (Applied Biosystems, Foster City, Calif.) in a total volume of 20 μl. Primers (exon-spanning) are pre-mixed with SYBR-Green JumpStart™ Taq ReadyMix™ (Sigma-Aldrich), and aliquots of 11 μl are applied to 96-well MicroAmp Optical plates (Applied Biosystems). cDNA is diluted 1:10, and aliquots of 2 μl are added in triplicates. Thermal cycling conditions are: 2 min at 50° C., 10 min at 95° C., and 40 cycles of 15 s at 95° C. and 1 min at 65° C. on an ABI Prism-7000 Sequence Detection Real-Time PCR System (Applied Biosystems). The ΔCt method is used to calculate relative changes in mRNA abundance. The threshold cycle (Ct) for TATA-binding protein (TBP) is subtracted from the Ct for the target gene to adjust for variations in the cDNA synthesis. miR-206 expression is determined as in a previous study (Walden et al. (2009) J. Cell Physiol. 218:444-449). TBP mRNA is used as endogenous control.

(d) Immuno Histochemistry: The harvested tissues are fixed by vascular perfusion of 1% paraformaldehyde in PBS are stained for hematoxylin and eosin (HE), embedded in paraffin, and sectioned at 5 μm and are whole-mounted for immunostaining (Koh et al., 2007). Whole-mount tissues are prepared and incubated for 1 h at room temperature with blocking solution containing 5% donkey serum (Jackson Immuno-Research laboratories Inc.) in PBST (0.3% Triton X-100 in PBS). After blocking, the whole-mounted tissues are incubated overnight at 4° C. with one or more of the following primary antibodies: (a) for lipid droplets of adipocytes, guinea pig anti-perilipin antibody (diluted 1:100; Acris Antibodies GmbH); (b) for basement membrane of individual adipocytes, rabbit anti-collagen IV antibody (diluted 1:50-1:500; Millipore, USA); (c) for uncoupling protein-1 (UCP-1), rabbit anti-UCP-1 antibody (diluted 1:1000; Abcam, USA); or (d) for PGC-1 \acute{α}, rabbit anti-PGC-1\acute{α} antibody (diluted 1:500; Calbiochem). After several washes in PBST, whole-mounted tissues are incubated for 1 h at room temperature with one or more secondary antibodies: (a) Cy3- or Cy5-conjugated antiguinea pig antibody (diluted 1:500; Jackson Immuno-Research Laboratories); (b) Cy3- or Cy5-conjugated antirabbit antibody (diluted 1:500; Jackson ImmunoResearch Laboratories). For special staining applied after the antibody incubations, whole mounted tissues are stained for 30 min at room temperature with one or more of the following: (a) for active mitochondria, MitoTracker Red CMXRos (MitoTracker, 100 nM in PBS; Invitrogen); (b) for nuclei, 4,6-diamidino-2-phenylindole, dihydrochloride (DAPI, 1 μg/ml in PBS; Invitrogen). For control experiments, the primary antibody is omitted or substituted with preimmune serum. Signals are visualized and digital images are obtained using a Zeiss ApoTome microscope and a Zeiss LSM 510 confocal microscope equipped with argon and helium-neon lasers (Carl Zeiss). For determining the unilocular and multilocular adipocytes, double-immunostained color images for perilipin and collagen IV are captured with a Zeiss LSM 510 confocal microscope. For determination of percentage of multilocular adipocytes in total adipocytes in the indicated adipose tissue, adipocytes are counted in 10 random regions (˜100 adipocytes/each region) per adipose tissue treated with rosiglitazone, and presented as a percentage of the total counted adipocytes. For determination of nuclei per adipocyte in the indicated adipose tissue, adipocytes are counted by 2 investigators in 10 regions (˜200 adipocytes/each region) per adipose tissue treated with indicated agents. For calculating the mitochondrial contents and UCP-1 expression, immunostained color images for MitoTracker or UCP-1 are captured with a Zeiss LSM 510 confocal microscope. Using ImageJ software (rsb.info.nih.gov/ij), the MitoTracker or UCP-1 area is selected as a region-of-interest from the images, and converted to 8-bit gray scale. Area densities of the MitoTracker or UCP-1-stained images are measured from the pixels in the region-of-interest; only pixels over a certain level (>50 intensity value) are taken to exclude background fluorescence.

(e) Western blotting: Freshly harvested adipose tissue is placed in Krebs-Ringer solution buffered with HEPES (KRH), pH 7.4, supplemented with 2.5% BSA, and finely minced with scissors. Enzymatic digestion is performed in KRH supplemented with 1 mg/ml collagenase type 1 and 2.5% BSA (pH 7.4), using shaking orbital bath for 30 min at 37° C. The undigested tissue is separated from isolated adipocytes by filtration through chiffon material. Isolated adipocytes are homogenized using a polytron homogenizer in ice-cold RIPA buffer (50 mM Tris_HCl, pH 7.4, 1% Triton X-100, 150 mM NaCl, 1 mM EDTA, 1 mM PMSF [protease inhibitor cocktail (Complete-Mini, Roche Diagnostics)], 1 mM Na₃VO₄, and 1 mM NaF). The homogenate is centrifuged at 14,000 g for 15 min.

The concentration of proteins in the supernatant is determined using the method of Lowry. An equal volume of reducing sample buffer [62.5 mM Tris-HCl, pH 6.8, 2% (wt/vol) SDS, 10% (vol/vol) glycerol, 100 mM dithiothreitol, and 0.1% (wt/vol) bromphenol blue] is added to each sample. Proteins are separated by SDS-PAGE. and transferred to polyvinylidene difluoride membranes (GE Healthcare Life Sciences) in 48 mM Tris-HCl, 39 mM glycine, 0.037 (wt/vol) SDS, and 15% (vol/vol) methanol using a semi-dry electrophoretic transfer cell (Bio-Rad Trans-Blot SD; Bio-Rad Laboratories) at 1.2 mA/cm2 for 90 min. After transfer, the membrane is stained with Ponceau S for examination of equal loading of proteins. After being washed, the membrane is blocked in 5% milk in Tris-buffered saline-Tween for 1 h at room temperature and probed with the indicated antibodies overnight at 4° C. The immunoblot is visualized with appropriate horseradish peroxidase-conjugated secondary antibodies and enhanced chemiluminescence (ECL kit, GE Healthcare Life Sciences) in a charge-coupled device camera (Fuji Film). UCP1 antibody (rabbit polyclonal, raised against C-terminal decapeptide), diluted 1:3000, and VDAC monoclonal antibody (Calbiochem, 529536) diluted 1:2000 is used.

### Example 3

Female mice (40 gm) from Jackson Labs (BKS.Cg-Dock7 m+/+Leprdb/J) are treated with pellets containing rosiglitazone maleate (obtained from Innovative Research of America, Sarasota, Fla. 34236 USA) at the release rates indicated in Table 2 and paramaters are measured as described in Example 2.

TABLE 2
EXPERIMENTAL DESIGN

| | | Number of | Amount of ATI-101 | Calculated |
|---|---|---|---|---|
| | Number of | pellets/ | released/pellet/ | Dose/animal/ |
| Group | animals | animal* | day at each site | day |
| 1 | 5 | 2 | 0 | 0 |
| (Placebo) | | | | |
| 2 | 5 | 2 | 100 ug | 200 ug |
| 3 | 5 | 2 | 1 ug | 2 ug |
| 4 | 5 | 2 | 0.1 ug | 0.2 ug |

*Pellets inserted on right side only (in the vicinity of inguinal pad) and one in the vicinity of fat pad in the interscapular space on the back of each mouse.

### Example 4

### Therapeutic Administration of Rosiglitazone

A 70 kg patient suffering from type 2 diabetes receives a subcutaneous administration into the abdominal area of a sustained release formulation of rosiglitazone optimized to release the drug over a 6-month period into the subcutaneous space to induce and maintain the modified morphology of the subcutaneous adipose tissue. The slow release of rosiglitazone from the sustained release formulation results in minimal systemic exposure of the drug, such that the plasma exposure (AUC₀₋₂₄ₕ) of rosiglitazone at steady state does not exceed about 300 ng-h/mL. The treatment results in an increase in subcutaneous adipose tissue mass, and alters its metabolic profile resulting in euglycemia or improved euglycemic control, thus delaying the need for other therapy, while minimizing the adverse effects such as cardiovascular effects, osteoporosis that are associated with oral dosing of rosiglitazone.

### Example 5

### Therapeutic administration of pioglitazone

A 70 kg patient suffering from type 2 diabetes receives a subcutaneous administration into the abdominal area of a sustained release formulation of pioglitazone optimized to release the drug over a 6-month period into the subcutaneous space to induce and maintain the modified morphology of the subcutaneous adipose tissue. The slow release of pioglitazone from the sustained release formulation results in minimal systemic exposure of the drug, such that the plasma exposure (AUC₀₋₂₄ₕ) of pioglitazone and its active metabolites at steady state does not exceed about 10 μg-h/mL. The treatment results in an increase in subcutaneous adipose tissue mass, and alters its metabolic profile resulting in euglycemia or improved euglycemic control, thus delaying the need for other therapy, while minimizing the adverse effects such as cardiovascular effects, osteoporosis that are associated with oral dosing of pioglitazone.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for treating a mammalian subject with a condition selected from the group consisting of type 2 diabetes, hyperlipidemia, and cardiovascular disease, wherein said method comprises:
administering a sustained release composition into a subcutaneous space in a localized area of said subject;
wherein said composition comprises a peroxisome proliferator-activated receptor (PPAR) agonist;
wherein said composition releases said agonist directly into said subcutaneous space;
wherein subcutaneous adipose tissue in said space is exposed in situ to a therapeutically effective amount of said agonist; and,
whereby systemic exposure in said subject to said agonist is minimized.

2. The method of claim 1 wherein said agonist is selected from the group consisting of rosiglitazone, ciglitazone, troglitazone, englitazone, pioglitazone, muraglitazar, ragaglitazar, naveglitazar, and mixtures thereof.

3. The method of claim 1 wherein said composition is formulated to release said agonist in a total daily amount that is in the range of about one tenth to about one thousandth of a therapeutically effective oral daily dose of said agonist, and wherein said total daily amount of said agonist provides a therapeutic effect substantially equivalent to that of said oral daily dose.

4. The method of claim 3 wherein said therapeutically effective amount elicits an effect selected from at least one of improved glycemic control, euglycemia, and an improved lipid profile.

5. The method of claim 1 wherein a ratio of said PPAR agonist concentration in said subcutaneous space to a steady-state plasma concentration of said PPAR agonist plus its active metabolites is in the range of about 2 to about 10000.

6. The method of claim 1 wherein said tissue is exposed to said agonist for a duration sufficient to detectably increase the amount of UCP-1 in said tissue and/or to detectably increase the oxygen uptake of said tissue.

7. The method of claim 1 wherein said agonist is administered at a dose at which an adverse side effect due to said PPAR agonist is substantially undetectable in said subject, wherein said side effect is selected from at least one of cardiovascular disease, osteoporosis, increased susceptibility for bone fracture, adipogenesis in bone marrow, bladder cancer, hepatitis, myocardial infarction, stroke, macular edema, fluid retention, cardiac hypertrophy, atherosclerosis, and congestive heart failure.

8. The method of claim 7 wherein said agonist is rosiglitazone or pioglitazone and wherein said side effect comprises cardiovascular disease, osteoporosis, increased susceptibility for bone fracture, fluid retention, and adipogenesis in bone marrow.

9. The method of claim 1 wherein said subject is able to achieve an average preprandial plasma glucose concentration in the range of about 72 mg per deciliter to about 108 mg per deciliter.

10. The method of claim 1 wherein said subject is able to achieve an average bedtime plasma glucose values between about 110 mg per deciliter to about 150 mg per deciliter.

11. The method of claim 1 wherein said subject is able to achieve a 2-hour postprandial blood glucose in the range of about 90 mg per deciliter to about 144 mg per deciliter.

12. The method of claim 1 wherein said subject is able to achieve an HbA_{1c} value less than about 7%.

13. The method of claim 1 wherein said sustained release composition comprises said agonist coated onto a biodegradable or non-biodegradable scaffold and wherein the scaffold is inserted into the subcutaneous space via a surgical procedure.

14. The method of claim 1 wherein said agonist is rosiglitazone, and wherein the amount of rosiglitazone released results in an AUC₀₋₂₄ₕ of rosiglitazone which does not exceed about 300 ng-h/mL in plasma of said subject.

15. The method of claim 1 wherein said agonist is pioglitazone, and wherein the amount of pioglitazone released results in an AUC₀₋₂₄ₕ of pioglitazone and its active metabolites which does not exceed about 10 μg-h/mL in plasma of said subject.

16. The method of claim 1 wherein said agonist is rosiglitazone and wherein said rosiglitazone is released at a rate of about 0.0001 μg per day to about 1000 μg per day.

17. The method of claim 1 wherein said agonist is pioglitazone and wherein said pioglitazone is released at a rate of about 0.0001 μg per day to about 10 mg per day.

18. The method of claim 1 wherein said PPAR agonist is a nonthiazolidinedione.

19. A method for preventing or treating type 2 diabetes and related disorders in a subject, the method comprising:
exposing subcutaneous adipose tissue of said subject in situ to a peroxisome proliferator-activated receptor agonist at a sufficient level and over a sufficient duration to activate brown adipocyte-like differentiation in said adipose tissue, wherein the differentiated subcutaneous adipose tissue has increased energy expenditure, wherein substantially no adverse effect due to said agonist is detectable in said subject.

20. A method comprising:
exposing subcutaneous adipose tissue in situ in a subject to a peroxisome proliferator-activated receptor agonist, wherein said exposing is sufficient to increase and maintain an increase in the quantity of brite adipocytes in said tissue.
